(19) United States
(12) Patent Application Publication
(10) Pub. No.: US 2020/0341000 A1
(43) Pub. Date: Oct. 29, 2020

(54) METHOD OF SCREENING COMPOUNDS FOR TREATING CNS DISORDERS

(71) Applicants: SAGE THERAPEUTICS, INC., Cambridge, MA (US); TRUSTEES OF TUFTS COLLEGE, Medford, MA (US)

(72) Inventors: Michael Andrew Ackley, Sudbury, MA (US); James J. Doherty, Bedford, MA (US); Stephen James Moss, Dover, MA (US); Manasa Lakshmi Parakala, Medford, MA (US); Paul A. Davies, Roslindale, MA (US)

(21) Appl. No.: 16/755,303

(22) PCT Filed: Oct. 12, 2018
(86) PCT No.: PCT/US2018/055661

§ 371 Date: Apr. 10, 2020

Related U.S. Application Data
(60) Provisional application No. 62/571,698, filed on Oct. 12, 2017.

(51) Int. Cl. G01N 33/68; G01N 33/50; G01N 33/94; G01N 33/74; G01N 27/40

(52) U.S. Cl. CPC G01N 33/6803; G01N 33/5008; G01N 33/9426; G01N 2333/705; G01N 27/40; G01N 2500/10; G01N 33/743

(57) ABSTRACT

Provided herein are screening methods and systems for the identification and evaluation of candidate GABAergic modulators. These candidate agents or compounds are useful for treating or preventing CNS-related disorders.

### FUNDING

This invention was made with U.S. government support under Grant No: R01-MH097446 awarded by the National Institute of Mental health and National Institute of Health and Grant Nos. W81XWH-15-1-0190 and W81XWH-15-1-0191 awarded by the United States Army. The U.S. government has certain rights in the invention.

### FIELD OF THE INVENTION

The present invention relates to methods and therapeutic agents for treating or preventing CNS-related disorders.

### BACKGROUND OF THE INVENTION

Brain excitability is defined as the level of arousal of an animal, a continuum that ranges from coma to convulsions, and is regulated by various neurotransmitters. In general, neurotransmitters are responsible for regulating the conductance of ions across neuronal membranes. Gamma-aminobutyric acid (GABA) is a major inhibitory neurotransmitter in the mammalian central nervous system (CNS). GABAergic inhibition refers to GABA-mediated neurotransmission which is inhibitory to mature neurons in vertebrates. Bernard C et al., Epilepsia (2000) 41(S6):S90-S95). Activation of GABA receptors by GABA causes hyperpolarization of neuronal membranes and a resultant inhibition of neurotransmitter release, thereby reducing brain excitability.

GABAergic inhibition is implicated in various CNS-related disorders, including but not limited to psychiatric and neurological conditions associated with impaired neuronal excitability, such as rapid mood changes, anxiety, stress response and epilepsy.

New and improved agents capable of modulating GABAergic inhibition are needed for the prevention and treatment of these CNS-related disorders. The methods and systems described herein are directed towards this end.

### SUMMARY OF THE INVENTION

The present invention is based, at least in part, on the surprising discovery that some membrane progesterone receptor (mPR) agonists modulate GABAergic inhibition, and are useful for treating CNS-related disorders. The modulation effect of these candidate GABAergic inhibitors can be allosteric, metabotropic, or both.

In one aspect, provided herein is a method of screening for a candidate GABA receptor trafficking modulator comprising the steps of a) contacting a test agent with a cell expressing at least one gamma-aminobutyric acid (GABA) receptor subunit; b) measuring a membrane-associated amount of the at least one GABA receptor subunit of the cell; c) comparing the membrane-associated amount of the at least one GABA receptor subunit in the cell contacted with the test agent with a membrane-associated amount of the at least one GABA receptor subunit in a cell not contacted with the test agent; and wherein if the membrane-associated amount in the cell contacted with the test agent is greater than the membrane-associated amount in the cell not contacted with the test agent, the test agent is a candidate GABA receptor trafficking modulator.

In some embodiments, measuring a membrane-associated amount of the at least one GABA receptor subunit of the cell comprises measuring: (1) an amount of the at least one GABA receptor subunit that is located on the cell membrane; (2) an amount of the at least one GABA receptor subunit that is incorporated into a GABA receptor; (3) a ratio between a membrane-associated amount of the at least one GABA receptor subunit and a soluble amount of the at least one GABA receptor subunit; (4) a rate of endocytosis of membrane-associated GABA receptors, or any combination of (1)-(4). In some embodiments, measuring a membrane-associated amount of the at least one GABA receptor subunit of the cell comprises performing a Western Blot assay, or an immunohistochemistry assay.

In some embodiments, the invention relates to a method of screening for a candidate GABA receptor trafficking modulator comprising the steps of: a) contacting a test agent with a cell expressing at least one gamma-aminobutyric acid (GABA) receptor subunit; b) measuring an expression level of the at least one GABA receptor subunit of the cell; c) comparing the expression level of the at least one GABA receptor subunit of the cell contacted with the test agent with an expression level of the at least one GABA receptor subunit of a cell not contacted with the test agent; and wherein if the expression level of the at least one GABA receptor subunit in the cell contacted with the test agent is greater than the expression level of the at least one GABA receptor subunit in the cell not contacted with the test agent, the test agent is a candidate GABA receptor trafficking modulator.

In some embodiments, measuring an expression level of the at least one GABA receptor subunit of the cell comprises measuring (1) a total amount of the at least one GABA receptor subunit in the cell; and/or (2) a total amount of a nucleic acid encoding the at least one GABA receptor subunit in the cell. In some embodiments, measuring an expression level of the at least one GABA receptor subunit of the cell comprises performing a Western Blot assay or a Northern Blot assay.

In some embodiments, the invention relates to a method of screening for a candidate GABA receptor trafficking modulator comprising the steps of: a) contacting a test agent with a cell expressing at least one gamma-aminobutyric acid (GABA) receptor subunit; b) measuring a phosphorylation level of the at least one GABA receptor subunit in the cell contacted with the test agent; c) comparing the phosphorylation level of the at least one GABA receptor subunit in the cell contacted with the test agent with a phosphorylation level of the at least one GABA receptor subunit in a cell not contacted with the test agent; and wherein if the phosphorylation level in the cell contacted with the test agent is greater than the phosphorylation level in the cell not contacted with the test agent, the test agent is a candidate GABA receptor trafficking modulator.

In some embodiments, the phosphorylation is protein kinase C (PKC)-mediated phosphorylation. In some embodiments, the phosphorylation level of an α4 GABA subunit, a β3 GABA subunit, or a combination thereof is measured. In some embodiments, the phosphorylation occurs at S408/409 of the β3 subunit and/or at S433 of the α4 subunit.

In some embodiments, measuring a phosphorylation level of the at least one GABA receptor subunit in the cell contacted with the test agent comprises measuring the phosphorylation level via a Western Blot assay employing an anti-phosphorylated subunit antibody.

In some embodiments, the at least one GABA receptor subunit is selected from a α1 subunit, a β2 subunit, a γ2 subunit, an α4 subunit, a β subunit, and a δ subunit, and any combination thereof. In some embodiments, the at least one GABA receptor subunit comprises a combination of α1β2γ2 subunits or a combination of α4β3δ subunits. In some embodiments, the GABA receptor is selected from a synaptic GABA receptor, an extrasynaptic GABA receptor, and a combination thereof In some embodiments, the synaptic GABA receptor comprises one or more subunits selected from an α1 subunit, a β2 subunit, and a γ2 subunit. In some embodiments, the extrasynaptic GABA receptor comprises one or more subunits selected from an α4 subunit, a β3 subunit, and a δ subunit.

In some embodiments, the at least one GABA receptor subunit is encoded by (1) an endogenous gene, (2) a heterologous gene, (3) an artificial expression construct; or (4) a combination thereof.

In some embodiments, the GABA receptor trafficking modulator is a natural or synthetic neuroactive steroid. In some embodiments, the GABA receptor trafficking modulator is a membrane progesterone receptor (mPR) modulator. In some embodiments, the GABA receptor trafficking modulator is a progesterone analog.

In some embodiments, the cell used in connection with the present methods is a brain cell. In some embodiments, the cell used in connection with the present methods is a dentate gyms granule cell (DGGC).

In some embodiments, the invention relates to a method of screening for a candidate GABA receptor trafficking modulator comprising the steps of: a) contacting a test agent with a cell expressing at least one membrane progesterone receptor (mPR); b) measuring an activity level of a mPR signaling pathway in the cell contacted with the test agent; c) comparing the activity level of the mPR signaling pathway in the cell contacted with the test agent with an activity level of the mPR signaling pathway in a cell not contacted with the test agent; wherein if the activity level of the mPR signaling pathway in the cell contacted with the test agent is greater than the activity level of the mPR signaling pathway in the cell not contacted with the test agent, the test agent is a candidate GABA receptor trafficking modulator. In some embodiments, the method further comprises d) measuring a binding affinity between the test agent and the mPR; and wherein if the binding affinity is above a predetermined threshold, the test agent is a candidate GABA receptor trafficking modulator.

In some embodiments, the greater activity level is indicated by an increase in protein kinase C (PKC) activity. In some embodiments, the greater activity level is indicated by an increase in PKC-mediated phosphorylation of at least one GABA receptor subunit in the cell. In some embodiments, the greater activity level is indicated by a reduced level of cellular cAMP. In some embodiments, the greater activity level is indicated by an increase in a gene expression level; wherein the gene encodes for at least one GABA receptor subunit or the gene is a reporter gene. In some embodiments, the gene is an endogenous gene or an artificial expression construct.

In some embodiments, the greater activity level is indicated by a higher membrane-associated amount of at least one GABA receptor subunit, the greater activity level is indicated by a greater GABAergic current in the cell. In some embodiments, the greater activity level is indicated by an increase in association between the mPR and a substrate.

In some embodiments, measuring an activity level of a mPR signaling pathway in the cell contacted with the test agent comprises measuring a level of (1) PKC activity; (2) PKC-mediated phosphorylation of at least one GABA receptor subunit; (3) cellular cAMP; (4) expression of a gene encoding for at least one GABA receptor subunit or a reporter gene; (5) a membrane-associated amount of at least one GABA receptor subunit; (6) GABAergic current in the cell, or any combination of (1)-(6).

In some embodiments, the method of screening for a candidate GABA receptor trafficking modulator further comprises a method of screening for a candidate GABA receptor potentiator, said method comprising: d) contacting the test agent with a membrane-associated gamma-aminobutyric acid (GABA) receptor; e) measuring a GABAergic current conducted by the GABA receptor of the membrane contacted with the test agent in the presence of GABA; f) comparing the GABAergic current of conducted by the GABA receptor contacted with the test agent with a GABAergic current conducted by the GABA receptor not contacted with the test agent; and wherein if the GABAergic current conducted by the GABA receptor contacted with the test agent is greater than the GABAergic current of conducted by the GABA receptor not contacted with the test agent, the test agent is a candidate GABA receptor potentiator.

In some embodiments, the GABA receptor is a cell membrane receptor. In some embodiments, the GABA receptor is a postsynaptic cell membrane receptor. In some embodiments, the GABA receptor is located within a synaptic area of the postsynaptic cell membrane. In some embodiments, the GABA receptor is located outside a synaptic area of the postsynaptic cell membrane.

In some embodiments, the GABAergic current is a tonic current and/or a spontaneous inhibitory post-synaptic current (sIPSC).

In some embodiments, a greater GABAergic current is indicated by: (1) a larger average amplitude of the tonic current; (2) a higher average current density of the tonic current; (3) a larger average amplitude of the sIPSC; (4) a longer average decay time of the sIPSC; or (5) any combination of (1)-(4).

### BRIEF DESCRIPTION OF THE DRAWINGS

FIG. 1 shows NASs (neuroactive steroids) allosterically modulate DGGC (dentate gyms granule cells) tonic currents. Panel (A) is a scheme demonstrating experimental protocol. Panel (B) is an example of current recordings from DGGCs showing modulation of tonic currents by acutely applied NASs (left panel). Bars above current recordings represent application of NAS and picrotoxin. Bar graphs summarizing the average tonic current (mean±SEM) before and during acute exposure with ALLO, SGE-516, or ganaxolone (right panel). *=significantly different to control (p=0.004; t-test), n=4-12 cells.

FIG. 2 shows allosteric modulation of phasic currents by acutely applied NASs. Recordings of sIPSCs from DGGCs before and during acute exposure to 100 nM ALLO, 100 nM SGE-516, or 100 nM ganaxolone for 10 min. Example of current recordings from DGGCs showing phasic currents before and during acutely applied NASs (left panel). Bar graphs summarizing the effects of acute exposure of ALLO, SGE-516, or ganaxolone on the amplitude and decay of sIPSCs (right panel). ***p=0.01, **p=0.03, *p=0.04, paired t-test, n=5-7 cells.

FIG. 3. shows NAS-mediated metabotropic enhancement of tonic inhibitory current in DGGC neurons. Panel (A) is a scheme demonstrating the experimental protocol. Left panel B, C, D show example tonic currents from slices following exposures to vehicle (control) or 100 nM ALLO (B), 100 nM SGE-516 (C), or 1 μM ganaxolone (D) for 15 min. No change in tonic current was observed in slices pre-incubated for 15 min with GFX followed by ALLO, or SGE-516. Bar above current represents application of picrotoxin (100 mM). Right panel B, C, D are bar graph showing that average tonic current was significantly enhanced following exposure to different concentrations of ALLO and SGE-516. No significant change in tonic current was observed following exposure to 1 μM ganaxolone for 15 min. In all panels *=significantly different to control (p<0.05; un-paired t-test, n=4-12 cells).

FIG. 4 shows glycine receptors do not contribute to tonic current in DGGCs. Hippocampal slices were incubated for 15 min with 100 nM ALLO or vehicle dissolved in ACSF then transferred to the recording chamber and washed for 30-60 min with NAS-free ACSF before recordings were started. Tonic current was measured by applying 100 μM picrotoxin in the absence or presence of the glycine receptor, strychnine (100 nM). Exposure to ALLO caused a significant increase in tonic current. Addition of strychnine did not alter the tonic current measured with picrotoxin. *p=0.01, unpaired t-test, n=4-12 neurons.

FIG. 5 shows sIPSC amplitude and decay was largely unchanged following exposure to NASs. Panel (A) shows IPSC recordings made from DGGCS in hippocampal slices from p21-35 C57 mice exposed to vehicle (control, n=6 neurons from 3 mice); panel (B) shows IPSC recordings made from DGGCS in hippocampal slices from p21-35 C57 mice exposed to 100 nM ALLO (n=4 neurons from, 2 mice); panel (C) shows IPSC recordings made from DGGCS in hippocampal slices from p21-35 C57 mice exposed to 100 nM SGE-516 (n=5 neurons from 2 mice); and panel (D) shows IPSC recordings made from DGGCS in hippocampal slices from p21-35 C57 mice exposed to 1 μM ganaxolone (n=5 neurons from 2 mice); for 15 min and then washed for >30 min prior to measurement of sIPSCs. Bar graphs show average sIPSC decay and amplitude. Only sIPSC amplitude was significantly enhanced following exposure to 100 nM SGE-516 but GFX (n=5 neurons from 2 mice) significantly reduced SGE-516 enhancement. *=significantly different to control (p<0.05; unpaired t-test).

FIG. 6. shows NAS exposure increases phosphorylation and surface expression of β3 subunits. Panel (A) shows exposure to 100 nM of the NASs, ALLO or SGE-516, for 20 min increases β3 S408/409 phosphorylation in acute hippocampal slices. Panel (B) shows the ratio of p-β3/T-β3 normalized to those in control (100%). Asterisks represent a significant difference from control (ALLO: p<0.01, Student's t-test, n=10 slices, from 10 mice; SGE-516: p<0.05, Student's t-test, n=4 slices, from 4 mice). Panel (C) shows exposure to 100 nM ALLO or SGE-516 for 20 min increases GABA_{A}-β3-containing receptors at the plasma membrane in acute hippocampal slices. Panel (D) shows the ratio of surface β3/T-β3 normalized to cell surface levels in control treated slices (100%). Asterisks represent a significant difference from control (ALLO: p<0.05, Student's t-test, n=8 slices; SGE-516: p<0.05, Student's t-test, n=4 slices).

FIG. 7 shows neurosteroids increase phosphorylation of GABA_{A}Rs and their cell surface stability. Panel (A) shows immunoblotting experiments of hippocampal slices treated with vehicle (Con), or SGE-516 for 20 min. The ratio of pS408/9/β3 immunoreactivity was normalized to control slices (100%=the line). Panel (B) shows the results of affinity purified pS443 used to immunoblot varying concentrations of the immunizing phosphor-peptide (PP). pS443 was used to immunoblot extracts of hippocampal slices treated without preadsorption (0), preadsorbed with the dephosphorylated (DP), or phosphorylated antigen (PP). Panel (C) shows immunoblotting experiments of hippocampal slices treated with vehicle (Con) or 100 SGE-516 for 5 min and then immunoblotted with pS443 and α4 antibodies as indicated. Panel (D) shows immunoblotting experiments of hippocampal slices treated as outlined above. Surface (S) and total (T) fractions were immunoblotted with α4 and β3 subunit antibodies. The ratio of S/T immunoreactivity and was normalized to control slices (100%=the line). Panel (E) shows the effect of diazepam (DZ) on cell surface stability of the β3 subunit. Panel (F) shows immunoblotting experiments of hippocampal extracts from C57/B16 mice injected with SGE-516 (5 mg/kg IP), or vehicle. SDS-soluble hippocampal extracts were immunoblotted with pS443, α4, pS408/9, or β3 subunit antibodies. In all panels: data represent mean±s.e.m.*=significantly different to control p<0.05 (t-test; n=7 mice).

FIG. 8 shows mutation of S408/9 in the β3 blocks the ability of SGE-516 to induce sustained effects on GABAergic inhibition. Panel (A) shows an experimental protocol used to examine the metabotropic effects of NASs on GABAergic currents. Panel (B) shows the sustained effects of SGE-516 on tonic currents measured in DGGCs from WT and S408/9A mice. Tonic current density was then compared between slices exposed to vehicle or SGE-516. *=significantly different control, p<0.05 (t-test; n=8 mice).

FIG. 9 shows that mutation of S408/9 in the β3 blocks the effects of SGE-516 on the cell surface levels of GABA_{A}Rs. Left panel (wide type) and right panel (S408/409A mutant) shows immunoblotting experiments of hippocampal slices treated as outlined above. Surface (S) and total (T) fractions were immunoblotted with α4 and β3 subunit antibodies. The ratio of S/T immunoreactivity and was normalized to control slices (100%=the line).

FIG. 10 shows measurements in the decay time of mIPSc in wild type (left panel) and S408/409A mutant (right panel) mice before and after treatment of ALLO.

FIGS. 11A and B show diagrams representing the protocols used to induced pharmacoresistant seizures in WT and S408/9A mice using kainate as measured using EEG recording.

FIGS. 12A and B show the ability of diazepam, SGE-516, THDOC in modifying seizure activity in S408/9 mice using EEG recording.

FIG. 13 shows % change in seizure power 10 minutes after treatment by diazepam, SGE-516, THDOC in wild type and S408/9A mutant mice.

FIG. 14 shows the diversity in ability of neuroactive steroids in modulating GABA receptor trafficking.

FIG. 15 shows that allopregnanolone (ALLO) and progesterone (P4) increase S408/9 phosphorylation in GT1-7 cells. Panel (A) upper section shows quantitative PCR analysis showing the enrichment of the mPRα mRNA in GT1-7 cells (taken from Thomas and Peng 2012). Lower section shows immunoblotting of 10 and 15 μg of SDS-soluble extracts from GT1-7 cells with an mPRα specific antibody. Panel (B) shows GT1-7 immunoblotting of cells treated with 100 nM ALLO or P4 for 15 min and immunoblotted with pS408/9, β3 and actin antibodies. The ratio of pS408/9/β3 subunit immunoreactivity were then normalized to levels seen in vehicle treated to controls, n=6.

FIG. 16 shows ALLO and ORG OD 02-0 induced sustained increases in GABA-evoked currents recorded from GT1-7 cells.

FIG. 17 shows that ORG OD 02-0(ORG) compound does not acutely modulate of the function of GABA_{A}Rs composed of α4β3 subunits. Upper panel shows sample traces of whole cell recording of GABA-induced currents (I_{GABA}) from cells treated with rapidly applied GABA (G), GABA and 100 nM ALLO (G&ALLO), or GABA and 100 mM ORG OD 02-0 (G&ORG). Lower panel shows the quantitation of percentage enhancement of I_{GABA} induced by the treatment.

FIG. 18 shows that P4 and ORG OD 02-0 regulated S408/9 phosphorylation in hippocampal slices. Panel (D) shows immunoblotting experiments of hippocampal slices were treated with 100 nM ALLO or P4 (progesterone), and S408/9 phosphorylation was then determined as detailed above, n=4 slices. Panel (E) shows immunoblotting experiments of Hippocampal slices were treated with 100 nM ORG OD 02-0, and S408/9 phosphorylation was examined using immunoblotting. In all panels; *=significantly different to control p<0.05 (one way ANOVA with Dunnet's multiple comparisons post-hoc test).

FIG. 19 shows dosage dependent effect of P4 and ORG OD 02-0 in modulating GABAergic tonic current. Left panel shows dosage-dependent effect of P4 and Org OD 02-0 in modulating amplitude of tonic current. Right panel shows dosage-dependent effect of P4 and ORG OD 02-0 in modulating density of tonic current.

FIG. 20 shows the mechanism of the mPR agonist-induced sustained elevations in GABAergic inhibition by promoting mPR-dependent phosphorylation of GABA_{A}Rs. Particularly, an mPR agonist such as a neuroactive steroids activates mPRs, which further activates protein kinase C (PKC), resulting in phosphorylation of GABA_{A}Rs on residues that include S408/9 in the β3 GABA receptor subunit. Enhanced phosphorylation for example at S408/9 results in enhanced trafficking of GABA_{A}Rs, an event that leads to a higher membrane density of GABA_{A}Rs, as well as a sustained increase in the efficacies of GABAergic phasic and tonic GABAergic inhibition.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Each embodiment of the invention described herein may be taken alone or in combination with one or more other embodiments of the invention.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Throughout this specification, the word “comprise” or variations such as “comprises” or “comprising” will be understood to imply the inclusion of a stated integer or groups of integers but not the exclusion of any other integer or group of integers. In each instance herein, in embodiments or examples of the present invention, any of the terms “comprising”, “consisting essentially of”, and “consisting of may be replaced with either of the other two terms in the specification. Also, the terms “comprising”, “including”, containing”, etc. are to be read expansively and without limitation.

The term “including” is used to mean “including but not limited to.” “Including” and “including but not limited to” are used interchangeably.

The singular forms “a,” “an,” and “the” include the plurals unless the context clearly dictates otherwise.

In order to further define the invention, the following terms and definitions are provided herein.

### Definitions

The term “GABAergic inhibition” refers to GABA (γ-Aminobutyric acid) mediated neurotransmission which is inhibitory to mature neurons in vertebrates. Bernard C et al., Epilepsia (2000) 41(S6):S90-S95).

GABA (γ-Aminobutyric acid) is a major inhibitory neurotransmitter in the spinal cord dorsal horn. GABA mediates both phasic and tonic inhibitory neurotransmission in the CNS through GABA_{A} receptors (GABA_{A}Rₛ). Particularly, GABA is released from presynaptic terminals of inhibitory neurons. Upon binding to GABA_{A} receptors at postsynaptic membrane, GABA elicits inhibitory postsynaptic currents (IPSCs). IPSCs provide phasic inhibition in neuronal network and are important for information processing. In addition to its action at synaptic sites, recent studies in several brain regions of matured animals have indicated that low concentrations of ambient GABA can activate high affinity GABA_{A} receptors that are expressed at extrasynaptic sites to elicit a sustained inhibitory current. Ataka et al. Mol Pain. 2006; 2: 36. The term “tonic inhibitory current” or “tonic current” has been used to describe this sustained inhibitory current. Functionally, tonic GABAergic inhibition has been shown to control neuronal excitability in the brain.

GABA_{A} receptors (GABA_{A}Rs) are heteropentameric ligand-gated ion channels that selectively permit the influx of Cl⁻ and HCO₃⁻ ions to decrease membrane excitability. Extremely heterologous with at least nineteen known subunit genes, GABA_{A} receptors mediate the majority of fast synaptic inhibition. GABA_{B} receptors (GABA_{A}Rs) are metabotropic G protein-coupled heterodimers of GABA-B1 and GABA-B2. They are expressed on both the presynaptic and postsynaptic terminals where they inhibit neurotransmitter release and induce cell membrane hyperpolarization, respectively. A third group of receptors was originally classified as GABA_{c} receptors (GABA_{C}Rs). These receptors are now considered as members of the GABA_{A} family.

GABA receptor subunits: GABA_{A}Rs are heteropentamers constructed from α(1-6), β(1-3), γ(1-3), δ, ε(1-3), θ, and/or π. subunits. There are thousands of possible subunit combinations, however only relatively few are expressed with any frequency in the mammalian central nervous system. Most GABA_{A}Rs are composed of 2α, 2β and 1γ (or 1δ) subunit. GABA_{A}Rs with different subunit composition have different physiological and pharmacological properties, are differentially expressed throughout the brain, and targeted to different subcellular regions. For instance, receptors composed of α(1,2,3 or 5) subunits together with β and γ subunits are largely synaptically located and mediate the majority of phasic inhibition in the brain (with the notable exception of extrasynaptically-localized α5-containing receptors). In contrast, those composed of α(4/6)βδ subunits form a specialized population of predominantly extrasynaptic receptor subtypes that mediate tonic inhibition. In addition, GABA_{A}Rs at presynaptic sites also exist. Jacob et al. Nat Rev Neurosci. 2008 May; 9(5): 331-343; Brickley S G, et al. Neuron 73(1):23-34.

Intracellular trafficking of GABA_{A} receptor: GABA_{A}Rs go through an intracellular trafficking cycle which begins with the assembly of the receptors in the endoplasmic reticulum (ER). After assembly in the ER, transport-competent GABA_{A}Rs are trafficked to the Golgi apparatus and segregated into vesicles for transport to, and insertion into, the plasma membrane, where they are able to access inhibitory postsynaptic specializations or extrasynaptic sites, depending on subunit composition. Membrane-associated GABA_{A}Rs undergo extensive endocytosis in both heterologous and neuronal systems. For example, approximately 25% of β3-containing cell surface GABA_{A}Rs being internalized within 30 minutes. Once endocytosed, most internalized GABA_{A}Rs recycle back to the plasma membrane over short time frames; however over longer time periods they are targeted for lysosomal degradation. Various protein factors are known to play a role in the intracellular trafficking of GABA_{A}Rs, including but are not limited to ubiquitin-proteasome system (UPS), ubiquitin-like proteins Plic-1 and Plic-2, N-ethylmaleimide-sensitive factor (NSF), GABA receptor-associated protein (GABARAP), Golgi-specific DHHC zinc finger domain protein (GODZ), BIG2, GRIF/TRAK proteins, Gephyrin, an ERM (ezrin, radixin, moesin)-family member protein, clathrin adaptor protein 2 (AP2) complex, and Huntingtin associated protein-1 (HAP1). Jacob et al. Nat Rev Neurosci. 2008 May; 9(5): 331-343.

The term “modulation” of an activity or physical state of a protein as used herein means increasing or decreasing an activity of that protein or a property of the protein's physical state resulting from contacting a test or candidate compound to a suitable test system. The modulation may be relative to another activity or property of a different protein, to the same protein in the basal state or subsequent to external stimulation, including contacting GABA to the test system prior to contacting of the testing agent, or relative to the change in activity or property from contacting the test system with vehicle or reference compound.

The term “modulator” of an activity or physical state of a protein as used herein refers to an agent or a composition comprising that agent which acts to increase or decrease the activity of that protein or property of the protein's physical state. For example, GABAergic modulators increase or decrease GABAergic inhibition in either an in vivo or in vitro setting.

The term “GABAergic modulator” as used herein refers to an agent which, upon being introduced to a test system, acts to modulate GABAergic inhibition via one or more mechanisms. Effect of a GABAergic modulator can be (1) allosteric, (2) metabotropic, or both.

The term “allosteric modulation” as used herein refers to the process of modulating a receptor by the binding of allosteric modulators at a site (i.e., regulatory site) other than that of the endogenous ligand (orthosteric ligand) of the receptor and enhancing or inhibiting the effects of the endogenous ligand. An allosteric modulator generally acts by causing a conformational change in a receptor molecule, which results in a change in the binding affinity of the ligand. Thus, an allosteric ligand (or modulator) modulates activation of a receptor by a primary “ligand” and can adjust the intensity of the receptor's activation. The effect of allosteric modulation is usually acute, arising immediately after exposing the test system to the allosteric modulator, and disappearing soon after the allosteric modulator is removed from the test system.

A “positive allosteric modulator (PAM)” enhances the effect of the endogenous ligand. For example, a PAM of GABA receptors typically interacts with the GABA receptor at a site different from the binding site of the orthosteric ligand—GABA, and enhances GABAergic inhibition. In some embodiments, allosteric modulation effect arises immediately after the test system is exposed to a modulator, and stops quickly after the modulator is removed from the system.

The term “metabotropic modulation” as used herein refers to the process of modulating a GABA receptor activity through signal transduction mechanisms. Metabotropic modulation can be sustained for a period of time after the metabotropic modulator has been removed from the test system.

Membrane progesterone receptors (mPRs) are G protein-coupled receptors belonging to the progestin and adipoQ receptor family (PAQR) that mediate a variety of rapid, cell surface-initiated progesterone action involving activation of intracellular signaling pathways. Human mPRs are classified into the following subtypes: mPRα (encoded by the PAQR7 gene), mPRβ (encoded by the PAQR8 gene), mPRγ (encoded by the PAQR5 gene), mPRδ (encoded by the PAQR6 gene), and mPRϵ (encoded by the PAQR9 gene). Analysis of mPR expression in various human tissues shows variable distribution of mPRα, mPRβ, and mPRγ. All three mPRs, mPRα, mPRβ, and mPRγ are found in human brain, including expression in the spinal cord, cerebral cortex, cerebellum, thalamus, pituitary gland, and caudate nucleus. Dressing et al. Steroids. 2011 January; 76(1-2): 11-17. The mPR can also bind to neurosteroids, such as progesterone and allopregnanolone. Thomas P, Pang Y (2012). Neuroendocrinology. 96 (2): 162-71. Petersen S L, et al. (2013). Frontiers in Neuroscience. 7: 164.

The term “signaling pathway” or “signal transduction pathway” as used herein refers to a sequence of biochemical events or the proteins and relay molecules involved in these events that transfer the consequence of a ligand binding event originating externally or internally to a cell or a cell-free system to an effector protein or receptor. The consequence (or signal) from these initial binding events are then transferred to another protein whose catalytic action or its effect on the catalytic action of another downstream protein amplifies the signal, which then may be passed along to yet another protein for further amplification to eventually modulate the activity or phosphorylation state of an effector protein or substrate terminal to the signal transduction cascade.

“Signal transduction node” as used herein refers to a component of a signal transduction pathway capable of having catalytic activity for incoming signal amplification. A signal transduction node may be an effector protein, protein complex, or non-protein component capable of this catalytic activity. The catalytic activity may be dependent upon the phosphorylation states of the effector protein, or one or more protein kinases that act upon them, or activities of effector molecules from other signal transduction pathways.

The term “phosphorylation status” or “phosphorylation state” or “phosphorylation level” as used herein interchangeably refers to the number or pattern of phosphate groups covalently bound to a phospho-protein, such as a phosphorylated GABA receptor subunit, which may be soluble, membrane bound and/or in a protein complex. For example, phosphorylation status may refer to the overall extent of phosphorylation of a collection of proteins for a specified protein complex or to the extent to which specified amino acid residue(s) of a specified protein in collection of such proteins that are capable of being phosphorylated are in fact phosphorylated. Protein Phosphorylation is typically catalyzed by protein kinases. Different protein kinases have different specificity and preference for substrates. For example, protein kinase C (PKC) is a family of protein kinases that are involved in controlling the function of other proteins through phosphorylation of hydroxyl groups of serine and threonine amino acid residues on these proteins.

“Test agent” as used herein refers to a candidate compound or a composition comprising the compound that is to be evaluated in a suitable test system for the presence or absence of one or more of the activities or effects being tested. For example, the effect of a test agent can be (1) allosteric, (2) metabotropic, or (3) both. A test agent also include reference agents whose effect on a suitable test system is known and which is to be compared with an effect (or lack thereof) provided by contacting another test agent to the same test system. A reference agent may serve as a positive or negative control for the identification and evaluation of candidate GABAergic modulators.

The term “cell-based system” as used herein refers to natural or artificial systems comprising one or more eukaryotic cellular components or synthetic counterparts thereof, which is constructed to facilitate screening and/or evaluating activities of candidate GABAergic modulators. The cell-based system encompasses whole cells cultured from an established cell lines or isolated from tissues, genetically modified cells, fractions or components of one or more types of cells, or any combination thereof. A cell-based system may further comprise artificial components such as an expression element and a reporter mechanism. For example, a cell-based system may express at least one GABA receptor subunit from an endogenous gene or an artificial genetic construct. A cell-based system may comprise one or more components that act as signal transduction nodes of a signaling pathway of interest. For example, a cell-based system may comprise components forming a membrane progesterone receptor (mPR)-mediated signaling pathway. A cell-based system may also comprise at least two types of cells of different functions, such as a presynaptic and a postsynaptic cell.

“Candidate GABA receptor trafficking modulator” as used herein refers to GABAergic modulators that affects the intracellular trafficking cycle of a GABA receptor. The modulation effect can be positive or negative. A positive modulation on the GABA receptor trafficking results in more functional GABA receptors in a test system, thus strengthening GABAergic inhibition. A negative modulation on the GABA receptor trafficking results in less functional GABA receptors in the test system, thus weakening GABAergic inhibition.

“A functional GABA receptor” as used herein refers to fully assembled GABA receptors that have been inserted into a membrane, thereby contributing to the electrical permeability of the membrane under the control of GABA, and other GABAergic modulators.

“Candidate GABA receptor potentiator” as used herein refers to GABAergic modulators that affects electrical permeability of a functional GABA receptor. Modulation by a candidate GABA receptor potentiator may be through the control of the open/close state of the ion channel formed by the GABA receptor. The term “open/close state” of an ion channel including whether the ion channel is open or closed at a given moment, the dimension of the opened channel at a given moment, the frequency of the ion channel becoming open in a given unit time, and/or the time duration of the ion change staying in the open state in a given unit time.

The term “neurosteroids” or “neuroactive steroid (NAS)” as used herein refers to a class of steroids, the natural forms of which are produced by cells of the central or peripheral nervous systems, independently of the steroidogenic activity of the endocrine glands. The neuroactive steroids as used herein can alter neuronal excitability through direct or indirect interaction with ligand-gated ion channels and/or other cell surface receptors. One class of neuroactive steroids are GABAergic modulators. Neuroactive steroid as used herein includes synthetic compounds, such as functional and/or structural analogs of natural neuroactive steroids.

A “subject” to which administration is contemplated includes, but is not limited to, humans (i.e., a male or female of any age group, e.g., a pediatric subject (e.g, infant, child, adolescent) or adult subject (e.g., young adult, middleaged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

### GABAergic Modulators

In one aspect of the present disclosure, provided herein are GABAergic modulators. According to the present disclosure, the candidate GABAergic modulators of this invention are useful in treating a CNS-related condition or disorder in a subject, including but not limited to, a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, a female sex dysfunction, and/or a neurodegenerative disease and/or disorder.

In some embodiments, GABAergic modulation can be via one or more different mechanisms. In some embodiments, the modulation effect can be (1) allosteric, (2) metabotropic or (3) both. As non-limiting examples, in some embodiments, the modulation is through increasing or decreasing the amount of GABA neurotransmitter release in a system. In some embodiments, the modulation is through increasing or decreasing the amount of functional GABA receptors present in a system. In some embodiments, the modulation is through potentiating or inhibiting GABA receptors in the system. As used herein, the system can be in an in vivo or in vitro setting, such as but not limited to a subject, a tissue extracted from the subject, a cell isolated or cultured, or a cell-based system.

In some embodiments, a metabotropic modulation results in a change in the amount of functional GABA receptors in the system. In some embodiments, a positive metabotropic GABAergic modulator acts to increase the overall amount of GABA receptors that are correctly assembled and inserted into cell membrane. In some embodiments, the positive metabotropic GABAergic modulator is a GABA receptor trafficking modulator.

In some embodiments, the GABA receptor trafficking modulator increases a membrane-associated amount of a GABA receptor by, for example, (1) increasing the level of expression of one or more GABA receptor subunits constituting the GABA receptor, (2) increasing the level of assembly of constituent GABA receptor subunits into the GABA receptor, (3) accelerating intracellular trafficking of the GABA receptor so that more copies of the receptors are trafficked to and inserted into the cell membrane; (4) increasing membrane stability of inserted copies of the GABA receptor so that the receptors stay functional for a longer period of time before endocytosed and recycled; or any combinations of mechanism (1) to (4).

In some embodiments, an allosteric modulation results in a change of potency of functional GABA receptors in the system. In some embodiments, a positive allosteric metabotropic GABAergic modulator acts to potentiate an existing functional GABA receptor. In some embodiments, the positive allosteric metabotropic GABAergic modulator is a GABA receptor potentiator.

In some embodiments, the GABA receptor potentiator increases electrical permeability of a GABA receptor by, for example, (1) increasing the frequency of the GABA receptor ion channel becoming open in a given unit time; (2) increasing the time duration of the GABA receptor ion channel stays open in a given unit time; (3) increasing the dimension of an opened GABA receptor ion channel; or any combinations of mechanism (1) to (3).

In some embodiments, a GABAergic modulator has both an allosteric and a metabotropic effect upon GABAergic inhibition. In some embodiments, a positive GABAergic modulator both increases the amount of functional GABA receptors in the system and potentiating existing GABA receptors. In some embodiments, a positive GABAergic modulator functions to (1) increasing the level of expression of one or more GABA receptor subunits constituting the GABA receptor, (2) increasing the level of assembly of constituent GABA receptor subunits into the GABA receptor, (3) accelerating intracellular trafficking of the GABA receptor so that more copies of the receptors are trafficked to and inserted into the cell membrane; (4) increasing membrane stability of inserted copies of the GABA receptor so that the receptors stay functional for a longer period of time before endocytosed and recycled; (5) increasing the frequency of the GABA receptor ion channel becoming open in a given unit time; (6) increasing the time duration of the GABA receptor ion channel stays open in a given unit time; (7) increasing the dimension of an opened GABA receptor ion channel; or any combinations of mechanism (1)-(7).

In some embodiments, the GABA receptor is a GABA_{A}R. In some embodiments, the GABA_{A}R receptor comprises at least one GABA receptor subunit or a functional domain thereof. In some embodiments, the GABA_{A}R receptor comprises at least one of α(1-6), β(1-3), γ(1-3), δ, ε(1-3), θ, and π subunits, or a functional domain thereof. In some embodiments, the GABA_{A}R receptor comprises at least one of α(1, 2, 3 or 5), β and γ subunits, or a functional domain thereof. In some embodiments, the GABA_{A}R receptor comprises at least one GABA receptor subunit selected from α(4/6), β, δ subunits, or a functional domain thereof. In some embodiments, the GABA_{A}R receptor comprises at least one GABA receptor subunit selected from 2α, 2β and 1γ (or 1δ) GABA receptor subunits, or one or more functional domains thereof. In some embodiments, the GABA_{A}R receptor comprises at least α1, β2 and γ2 GABA receptor subunits, or one or more functional domains thereof. In some embodiments, the GABA_{A}R receptor comprises at least α4, β3 and δ GABA receptor subunits, or one or more functional domains thereof.

In some embodiments, the GABAergic modulator is an agonist of a membrane progesterone receptor (mPR). In some embodiments, the mPR is mPRα. In some embodiments, the mPR is mPRβ. In some embodiments, the mPR is mPRγ. In some embodiments, the mPR is mPRδ. In some embodiments, the mPR is mPRϵ.

In some embodiments, the GABAergic modulator is a neuroactive steroid. In some embodiments, the neuroactive steroid is a natural compound. In some embodiments, the neuroactive steroid is a synthetic compound. In some embodiments, the neuroactive steroid is progesterone, a metabolite or a functional analog thereof In some embodiments, the neuroactive steroid is a compound selected from the table below:

| Name | Structure |
|---|---|
| Progesterone (P or P4) | 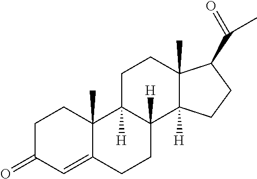 |
| Allopregnanolone (ALLO) | 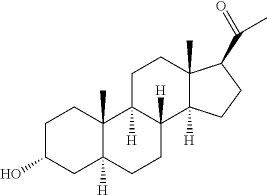 |
| Tetrahydrodeoxy- corticosterone (THDOC) | 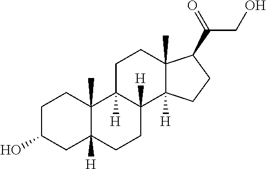 |
| Ganaxolone | 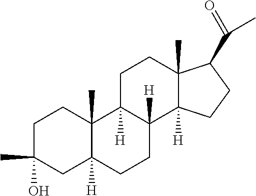 |
| 5α-Dihydropro- gesterone (5α-DHP) | 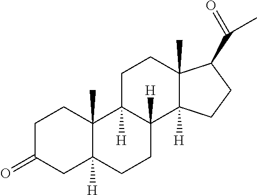 |

In some embodiments, the GABAergic modulator, upon binding to mPR, activates one or more downstream effector molecule in the mPR mediated signal transduction pathway in the system. In some embodiments, the GABAergic modulator activates heterotrimeric G proteins, which consists of three subunits, Gα, Gβ, and Gγ. When a G protein-coupled receptor (GPCR) is activated, Gα dissociates from Gβγ, allowing both subunits to perform their respective downstream signaling effects. In some embodiments, the GABAergic modulator affects the cellular cAMP level. In some embodiments, activated Gαi subunit inhibits the production of cAMP from ATP. In some embodiments, activated Gαi subunit increases the production of cAMP from ATP. In some embodiments, the GABAergic modulator inhibits the production of cAMP from ATP. In some embodiments, the GABAergic modulator regulates the activity level of cAMP response element-binding protein (CREB). In some embodiments, activated Gβγ subunit activates phospholipase C (PLC). In some embodiments, activated Gβγ subunit activates phosphoinositide 3-kinase (PI3K). In some embodiments, the GABAergic modulator activates phosphoinositide 3-kinase (PI3K).

PI3Ks are a family of related intracellular signal transducer enzymes capable of phosphorylating the 3 position hydroxyl group of the inositol ring of phosphatidylinositol (PtdIns). In some embodiments, activated PI3K further activates protein kinase C (PKC). In some embodiments, the GABAergic modulator activates kinase C (PKC).

Phospholipase C (PLC) is a class of membrane-associated enzymes that cleave phospholipids just before the phosphate group. In some embodiments, activated PLC further result in production of diacylglycerol (DAG) and inositol 1,4,5-trisphosphate (IP3). In some embodiments, accumulation of DAG and IP3 in the system further stimulates downstream signaling pathways that activate PKC and intracellular Ca²⁺ mobilization. In some embodiments, the GABAergic modulator increases cellular DAG and/or IP3 level. In some embodiments, the GABAergic modulator increases the amount of mobilized intracellular Ca²⁺.

In some embodiments, immobilization of intracellular Ca²⁺ further activates Ca2+/calmodulin-dependent protein kinase II (CaMKII), which is a serine/threonine-specific protein kinase. In some embodiments, the GABAergic modulator activates Ca2+/calmodulin-dependent protein kinase II (CaMKII).

In some embodiments, the GABAergic modulator activates one or more protein kinases. In some embodiments, one or more activated protein kinases increases phosphorylation level of one or more GABA receptor subunits. In some embodiments, the increased phosphorylation level of the one or more GABA receptor subunits further increase membrane stability of a GABA receptor constituted of the one or more GABA receptor subunits.

In some embodiments, the GABAergic modulator activates protein kinase C. In some embodiments, the GABAergic modulator activates CaMKII. In some embodiments, the GABAergic modulator activates protein kinase A (PKA). In some embodiments, the GABAergic modulator activates the proto-oncogene tyrosine-protein kinase Src. In some embodiments, the GABAergic modulator activates the mitogen-activated protein kinases (MAPK; also known as extracellular signal-regulated kinase (ERK)).

In some embodiments, the GABAergic modulator increases phosphorylation level of a α4 subunit. In some embodiments, the GABAergic modulator increases phosphorylation level of a β3 subunit. In some embodiments, the GABAergic modulator increases phosphorylation level of a α4 subunit at S443 position. In some embodiments, the GABAergic modulator increases phosphorylation level of a β3 subunit at S408/9 positions. In some embodiments, the GABAergic modulator increases phosphorylation level of β1 GABA receptor subunit. In some embodiments, the GABAergic modulator increases phosphorylation level of β2 GABA receptor subunit. In some embodiments, the GABAergic modulator increases phosphorylation level of γ2 GABA receptor subunit.

In some embodiments, the GABAergic modulator, upon binding to mPR, upregulates the expression level of at least one GABA receptor subunit. In some embodiments, the upregulated GABA receptor subunit is selected from α(1-6), β(1-3), γ(1-3), δ, ε(1-3), θ, and π subunits. In some embodiments, the upregulated GABA receptor subunit is selected from α(1, 2, 3 or 5), β and γ subunits. In some embodiments, the upregulated GABA receptor subunit is selected from α(4/6), β, δ subunits. In some embodiments, the upregulated GABA receptor subunits comprise 2α, 2β and 1γ(or 1δ) GABA receptor subunits. In some embodiments, the upregulated GABA receptor subunits comprise α1, β2 and γ2 subunits. In some embodiments, the upregulated GABA receptor subunits comprise α4, β3 and δ subunits.

In some embodiments, the GABAergic modulator, upon binding to mPR, upregulates the level of assembly of at least one GABA receptor. In some embodiments, the upregulated GABA receptor is trafficked to and inserted into a synaptic area of a cell membrane. In some embodiments, the upregulated GABA receptor is trafficked to and inserted into an extrasynatpic area of a cell membrane.

### Screening Methods

In one aspect, provided herein are methods of screening test agents for the identification of a GABAergic modulator. In some embodiments, the GABAergic modulator identified by the present screening method is a GABA receptor trafficking modulator. In some embodiments, the GABAergic modulator identified by the present screening method is a GABA receptor potentiator. In some embodiments, the GABAergic modulator identified by the present screening method is both a GABA receptor trafficking modulator and a GABA receptor potentiator.

According to the present disclosure, the candidate GABAergic modulators of this invention are useful in treating a CNS-related condition or disorder in a subject, including but not limited to, a psychiatric disorder, a neurological disorder, a seizure disorder, a neuro-inflammatory disorder, a sensory deficit disorder, pain, a neurodegenerative disease and/or disorder, a neuroendocrine disorder and/or dysfunction, a female sex dysfunction, and/or a neurodegenerative disease and/or disorder.

### Identifying a GABA Receptor Trafficking Modulator

In some embodiments, provided herein are methods for screening for a candidate GABA receptor trafficking modulator. The method comprises contacting a test agent with a suitable test system; measuring a selected test parameter from the test system contacted with the test agent; comparing the test parameter in the system contacted with the test agent with the test parameter in a system not contacted with the test agent; wherein if the test parameter in the system contacted with the test agent is greater than the test parameter in the system not contacted with the test agent, the test agent is a candidate GABA receptor trafficking modulator.

In some embodiments, the test system is a cell-based system, such as a whole cell or a system comprising fractions or components of one or more types of cells.

In some embodiments, the method of screening for a candidate GABA receptor trafficking modulator comprises measuring the amount of functional GABA receptors presented in the test system contacted with the test agent. In some embodiments, a functional GABA receptor comprises at least one GABA receptor subunit or a functional domain thereof. In some embodiments, a functional GABA receptor comprises at least one of α(1-6), β(1-3), γ(1-3), δ, ε(1-3), θ, and π subunits, or a functional domain thereof. In some embodiments, the functional GABA receptor comprises at least one of α(1, 2, 3 or 5), β and γ subunits, or a functional domain thereof. In some embodiments, the functional GABA receptor comprises at least one GABA receptor subunit selected from α(4/6), β, δ subunits, or a functional domain thereof. In some embodiments, the functional GABA receptor comprises at least one GABA receptor subunit selected from 2α, 2β and 1γ (or 1δ) GABA receptor subunits, or one or more functional domains thereof. In some embodiments, the functional GABA receptor comprises at least α1, β2 and γ2 GABA receptor subunits, or one or more functional domains thereof. In some embodiments, the functional GABA receptor comprises at least α4, β3 and δ GABA receptor subunits, or one or more functional domains thereof.

In some embodiments, the amount of functional GABA receptors in the test system can be measured by a membrane-associated amount of the GABA receptor, or a membrane-associated amount of a constituent subunit of the GABA receptor.

The phrases “membrane-associated amount of a GABA receptor” and “amount of membrane-associated GABA receptor” are used interchangeably to refer to the amount of a properly assembled GABA receptor that has been inserted into a cell membrane and become functional in conducting transmembrane GABAergic current. The term “membrane-associated amount” of a GABA receptor subunit refers to the amount of the subunit protein that is included in a membrane-associated and functional GABA receptor.

In some embodiments, a membrane-associated GABA receptor isolated from the test system as a multi-subunit protein complex and the amount is quantitated. In some embodiments, at least one membrane-associated subunit constituting the GABA receptor is isolated from the test system and the amount is quantitated.

In some embodiments, the membrane-associated amount of GABA receptor or GABA receptor subunit in the test system can be measured as a ratio between a membrane-associated amount of a target GABA receptor subunit and a soluble amount of the target GABA receptor subunit in the test system. The term “soluble amount” of a GABA receptor subunit refers to the amount of the subunit protein that is not associated with a membrane in the test system. In some embodiments, when the test system is a cell, the “soluble amount” of a GABA receptor subunit refers to the amount of the subunit protein that is present in the cytoplasm of a cell, regardless whether the GABA receptor subunit has been assembled into a GABA receptor. In some embodiments, the soluble amount of a target GABA receptor subunit can be measured as the difference between the total amount of the target GABA receptor subunit and the membrane-associated amount.

In some embodiments, the membrane-associated amount of GABA receptor or GABA receptor subunit in the test system can be measured as a ratio between a membrane-associated amount of a target GABA receptor subunit and a total amount of the target GABA receptor subunit in the test system. Various methods and techniques known in the art can be used to isolate and purify a target GABA receptor or subunit from a test system, which include but are not limited to, immunoprecipitation, density gradient centrifugation chromatography technologies, electrophoresis technologies, biotinylation assays. For example, quantitation can be achieved by western blotting. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the membrane-associated amount of GABA receptor or GABA receptor subunit is quantitated in situ without the need of their isolation and purification from the test system. Various methods and techniques known in the art can be used for in situ quantitation. For illustrative purpose only, in one example, in situ quantitation can be achieved by an immunohistochemistry assay. In some embodiments, the membrane and membrane-associated GABA receptor or GABA receptor subunit are immuno-stained with a fluorescent signal, and fluorescence intensity is quantified. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the membrane-associated amount of GABA receptor or GABA receptor subunit in the test system can be determining by measuring the level of assembly of at least two GABA receptor subunits. In some embodiments, the membrane-associated amount of GABA receptor or GABA receptor subunit in the test system can be measured as the amount of at least one GABA receptor subunit that is incorporated into a GABA receptor. In some embodiments, the amount of membrane-associated GABA receptor or GABA receptor subunit in the test system can be determined by measuring the level of association between a GABA receptor subunit and a membrane-associated protein that is not a GABA receptor subunit. Various methods and techniques known in the art can be used for detecting protein-protein interaction. For illustrative purpose only, in some embodiments, a specific antibody can be used to immunoprecipitate one target GABA receptor subunit, and then the amount of one or more other GABA receptor subunits or one or more other membrane-associated proteins associated with the target GABA receptor subunit can be quantitated by western blotting. In other embodiments, the level of assembly may be measured in situ as the distance between at least two GABA receptor subunits or between a GABA receptor subunit and another membrane-associated protein. Various methods and technique can be used to determine the distance between two proteins, such as fluorescence resonance energy transfer (FRET) and fluorescence quenching assay. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the membrane-associated amount of GABA receptor or GABA receptor subunit can be determining by measuring membrane stability of a functional GABA receptor in the test system. The term “membrane stability” refers to the measurement of the average time period that a membrane-associated receptor stays functional on the cell membrane before it is removed, degraded or otherwise inactivated.

During the intracellular trafficking cycle, membrane-associated GABA receptors are removed from the cell membrane via endocytosis. Thus, in some embodiments, membrane stability of a target GABA receptor is measured as the rate of endocytosis of the target GABA receptor. Various methods and techniques known in the art may be used to measure the rate of endocytosis of membrane-associated GABA receptors. For illustrated purpose only, in some embodiments, membrane-associated GABA receptors may be labeled or visualized by attaching a signal molecule to the membrane-associated GABA receptor. The signal or visualization remains only when the labeled GABA receptor remains on the membrane, and disappears once the GABA receptor is endocytosed. In some embodiments, the rate of endocytosis of the GABA receptor can be monitored by measuring the life span of the labeling signal. In some embodiments, the rate of endocytosis is determined by measuring activity of one or more protein factors mediating endocytosis of GABA receptors. In some embodiments, the protein factors mediating endocytosis of GABA receptors comprise clathrin adaptor proteins. In some embodiments, the rate of endocytosis of membrane-associated GABA receptors can be measured in the rate of Golgi transportation. In some embodiments, the rate of endocytosis of membrane-associated GABA receptors can be measured in the rate of vesicular transportation at synaptic terminals. In some embodiments, the rate of endocytosis of membrane-associated GABA receptors can be measured as the level of activity associated with lysosomal degradation of endocytosed receptors. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the method of screening for a candidate GABA receptor trafficking modulator comprises measuring the level of expression of at least one GABA receptor subunit in the test system contacted with the test agent. In some embodiments, the test system comprises one or more genetic elements encoding for at least one GABA receptor subunit. Various methods and techniques known in the art can be used to determine the level of expression of the at least one GABA receptor subunit. For illustrative purpose only, in one embodiment, the level of expression of a target GABA receptor subunit can be measured as the total amount of the target GABA subunit protein present in the test system. In another embodiment, the level of expression of a target GABA receptor subunit can be measured as the total amount of mRNA transcript of the encoding genetic element present in the test system. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the test system comprises a reporter mechanism capable of producing a signal indicative of the level of expression of a target GABA receptor subunit. Various methods and techniques known in the art can be used to provide the reporting mechanism in the test system. For illustrative purpose only, in one embodiment, the test system comprises a reporter gene comprising a coding sequence for a reporter protein operably linked to a regulatory element of a GABA gene. Thus, the level of expression of the signal peptide corresponds to an expected level of expression of a GABA receptor subunit encoded by the GABA gene. In some embodiments, the reporter protein is a fluorescent protein. In some embodiments, the reporter protein is a luciferase. In some embodiments, the reporter protein comprises a purification tag that facilitates the isolation of the reporter protein from the test system and subsequent quantitation. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the rate of endocytosis of a GABA receptor is affected by the phosphorylation status of one or more receptor subunits constituting the receptor. Thus, in some embodiments, the amount of functional GABA receptor in the test system is determined by measuring the phosphorylation status of the target GABA receptor or GABA receptor subunit. In some embodiments, the phosphorylation status is measured as the amount of phosphorylated target subunit in the system. In some embodiments, the phosphorylation status is measured as a ratio between phosphorylated target subunit and non-phosphorylated target subunit in the system. In some embodiments, phosphorylation of β3 subunit at S408/409 is measured. In some embodiments, phosphorylation of α4 subunit at S443 is measured. Various methods and techniques known in the art can be used to detect and quantify the amount of a protein in its phosphorylated state. For illustrative purpose only, in some embodiments, an antibody specifically recognizing a phosphorylated epitope in a target GABA receptor subunit can be used in a western blot assay to detect and quantify phosphorylated GABA receptor subunit. In some embodiments, the level of phosphorylation is measured by ELISA assay, a mass spectrometry assay, or an assay measuring kinase activity using radio-labels. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the rate of endocytosis of a GABA receptor is affected by phosphorylation mediated by protein kinase C (PKC). Thus, in some embodiments, the method of screening for a candidate GABA receptor trafficking modulator comprises measuring the activity of protein kinase C (PKC) in the test system contacted with the test agent. In some embodiments, the rate of endocytosis of a GABA receptor is affected by phosphorylation mediated by Ca2+/calmodulin-dependent protein kinase II (CaMKII). Thus, in some embodiments, the method of screening for a candidate GABA receptor trafficking modulator comprises measuring the activity of CaMKII in the test system contacted with the test agent. Various methods and techniques known in the art can be used to measure activity of target protein kinase in the test system. For illustrative purpose only, in some embodiments, the test system may comprise a PKC-selective peptide substrate or a CaMKII-selective peptide substrate, and phosphorylation of this substrate is measured. Chakravarthy et al. Analytical Biochemistry Volume 196, Issue 1, July 1991, Pages 144-150. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, a higher amount of functional GABA receptors in the test system is indicated by (1) a higher expression level of at least one GABA receptor subunit; (2) a higher level of assembly of the at least one GABA receptor subunit into a GABA receptor; (3) a lower endocytosis rate of a GABA receptor; (4) a higher membrane stability of a GABA receptor; (5) a higher membrane-associated amount of a GABA receptor or GABA receptor subunit; (6) a higher phosphorylation level of at least one GABA receptor subunit; (6) a higher activity level of PKC or CaMKII; and any combination of (1)-(7).

In some embodiments, the GABAergic modulator is an agonist of a membrane progesterone receptor (mPR). In some embodiments, the mPR is mPRα. In some embodiments, the mPR is mPRβ. In some embodiments, the mPR is mPRγ. In some embodiments, the mPR is mPRδ. In some embodiments, the mPR is mPRϵ.

In some embodiments, the method of screening for a candidate GABA receptor trafficking modulator comprises measuring an activity level of an mPR signaling pathway in the test system contacted with the test agent. In some embodiments, the activity level of the mPR signaling pathway is measured as the activity level of one or more signal transduction nodes comprised in the signaling pathway.

In some embodiments, the activity level of the mPR signaling pathway is measured as the activity level of heterotrimeric G proteins in the test system. In some embodiments, the activity level of the mPR signaling pathway is measured as the activity level of Gαi subunit of the heterotrimeric G proteins in the test system. In some embodiments, the activity level of the mPR signaling pathway is measured as the activity level of Gβγ subunit of the heterotrimeric G proteins in the test system. In some embodiments, the activity level of the mPR signaling pathway is measured as the activity level of phospholipase C (PLC) in the test system. In some embodiments, the activity level of the mPR signaling pathway is measured as the activity level of phosphoinositide 3-kinase (PI3K) in the test system. In some embodiments, the activity level of the mPR signaling pathway is measured as the activity level of PKC in the test system. In some embodiments, the activity level of the mPR signaling pathway is measured as the activity level of CaMKII in the test system. In some embodiments, the activity level of the mPR signaling pathway is measured as the activity level of protein kinase A (PKA) in the test system. In some embodiments, the activity level of the mPR signaling pathway is measured as the activity level of the proto-oncogene tyrosine-protein kinase Src. In some embodiments, the activity level of the mPR signaling pathway is measured as the activity level of the mitogen-activated protein kinases (MAPK; also known as extracellular signal-regulated kinase (ERK)). In some embodiments, the activity level of the mPR signaling pathway is measured as the amount of cAMP in the test system. In some embodiments, the activity level of the mPR signaling pathway is measured as the activity level of cAMP response element-binding protein (CREB). In some embodiments, the activity level of the mPR signaling pathway is measured as the amount of diacylglycerol (DAG) in the system. In some embodiments, the activity level of the mPR signaling pathway is measured as the amount of inositol 1,4,5-trisphosphate (IP3) in the system. In some embodiments, the activity level of the mPR signaling pathway is measure as the amount of mobilized Ca²⁺ in the test system. In some embodiments, the activity level of the mPR signaling pathway is measured as the affinity between mPR and a substrate downstream of the mPR mediated signaling pathway.

In some embodiments, the activity level of the mPR signaling pathway is measured as the phosphorylation state of at least one GABA receptor subunit in the test system. In some embodiments, the at least one GABA receptor subunit is a α4 subunit. In some embodiments, the at least one GABA receptor subunit is a β3 subunit. In some embodiments, the activity level of the mPR signaling pathway is measured as the phosphorylation state of the α4 GABA receptor subunit at S443 position. In some embodiments, the activity level of the mPR signaling pathway is measured as the phosphorylation state of the β3 GABA receptor subunit at S408/9 positions. In some embodiments, the at least one GABA receptor subunit is a β1 subunit. In some embodiments, the at least one GABA receptor subunit is a β2 subunit. In some embodiments, the at least one GABA receptor subunit is a γ2 subunit.

In some embodiments, the activity level of the mPR signaling pathway is measured as a level of expression of a GABA receptor subunit in the test system. In some embodiments, the activity level of the mPR signaling pathway is measured as a level of expression of a reporter gene in the test system, the expression level of the reporter indicative of an expected expression level of at least one GABA receptor subunit. In some embodiments, the sequence coding for the GABA receptor subunit is part of an endogenous gene. In some embodiments, the sequence coding for the GABA receptor subunit is part of an artificial genetic construct. Various artificial genetic constructs known in the art can be used in connection with the present invention.

In some embodiments, the activity level of the mPR signaling pathway is measured as a membrane-associated amount of at least one GABA receptor or a constituent GABA receptor subunit.

In some embodiments, the activity level of the mPR signaling pathway is measured as total electrical permeability of a membrane having at least one GABA receptor in the system. In some embodiments, the electrical permeability of the membrane is determined by measuring a total GABAergic current from the test system. Various methods and techniques known in the art can be used for measuring electrical permeability of the membrane. For illustrative purpose only, in some embodiments, the GABAergic current is measure by electrophysiological recording methods, including but not limited to whole-cell recording, perforated patch recording, single-unit recording, multi-unit recording, patch clamp recording, voltage-clamp recording, current-clamp recording, field potential measurement, amperometry measurement. After consulting the present disclosure, one skilled in the art may envisage numerous other changes, substitutions, variations, alterations, and modifications without inventive activity, and it is intended that the present disclosure encompasses all such changes, substitution, variations, alteration, and modifications as falling within its scope.

In some embodiments, the method of screening for a candidate GABA receptor trafficking modulator further comprises measuring a binding affinity between the test agent and mPR, and comparing the binding affinity to a pre-determined threshold. In some embodiments, if the binding affinity is above the predetermined threshold, the test agent is considered as a candidate GABA receptor trafficking modulator.

In some embodiments, a higher level of activity of the mPR-mediated signal pathway is indicated by (1) a higher activity of heterotrimeric G proteins; (2) a higher activity level of the Gαi subunit of the heterotrimeric G proteins; (3) a higher activity level of Gβγ subunit of the heterotrimeric G proteins; (4) a higher activity level of phospholipase C (PLC); (5) a higher activity level of protein kinase A (PKA); (6) a higher activity level of the proto-oncogene tyrosine-protein kinase Src; (7) a higher activity level of the mitogen-activated protein kinases (MAPK; also known as extracellular signal-regulated kinase (ERK)); (8) a higher activity level of PI3K; (9) a higher activity level of PKC; (10) a higher activity level of CaMKII; (11) a lower amount of cAMP; (12) a lower activity level of CREB; (13) a higher amount of diacylglycerol (DAG); (14) a higher amount of inositol 1,4,5-trisphosphate (IP3); (15) a higher amount of mobilized Ca²⁺; (16) a higher affinity between mPR and a substrate downstream of the mPR mediated signaling pathway; (17) a higher phosphorylation level of at least one GABA receptor subunit; (18) a higher level of expression of a GABA receptor subunit; (19) a higher membrane-associated amount of GABA receptor or a GABA receptor subunit; (20) a higher electrical permeability of a membrane having at least one GABA receptor; or any combination of (1)-(20).

### Identifying a GABA Receptor Potentiator

In some embodiments, provided herein are methods for screening for a candidate GABA receptor potentiator. The method comprises contacting a test agent with a suitable test system; measuring a selected test parameter from the test system contacted with the test agent; comparing the test parameter in the system contacted with the test agent with the test parameter in a system not contacted with the test agent; wherein if the test parameter in the system contacted with the test agent is greater than the test parameter in the system not contacted with the test agent, the test agent is a candidate GABA receptor potentiator.

In some embodiments, the test system comprises at least one membrane associated GABA receptor. In some embodiments, the at least one membrane associated GABA receptor is located on a cell membrane. In some embodiments, the at least one membrane associated GABA receptor is part of a cell-based system. In some embodiments, the at least one membrane associated GABA receptor is located on a postsynaptic membrane. In some embodiments, the at least one membrane associated GABA receptor is located in a synaptic region of the postsynaptic membrane. In some embodiments, the at least one membrane associated GABA receptor is located in an extrasynaptic region of the postsynaptic membrane. In some embodiments, the at least one membrane associated GABA receptor is located on a partial cell membrane with a solid support. In some embodiments, the at least one membrane associated GABA receptor is located on an artificial membrane.

In some embodiments, the screening method comprises screening test agents for the identification of a candidate GABA receptor potentiator. The method comprises the steps of:

a) contacting the test agent with at least one membrane-associated GABA receptor;

b) measuring a GABAergic current conducted by the GABA receptor in the presence of GABA;

c) comparing the GABAergic current conducted by the GABA receptor contacted with the test agent with a GABAergic current conducted by the GABA receptor not contacted with the agent; and

wherein if the GABAergic current conducted by the GABA receptor contacted with the test agent is greater than the GABAergic current conducted by the GABA receptor not contacted with the test agent, the test agent is a candidate GABA receptor potentiator.

In some embodiments, the GABA receptor is on a cell membrane. In some embodiments, the GABA receptor is on a postsynaptic cell membrane. In some embodiments, the GABA receptor is located within the synaptic area of the postsynaptic cell membrane. In some embodiments, the GABA receptor is located in the extrasynaptic area of the postsynaptic cell membrane. In some embodiments, the GABA receptor conducts a tonic current. In some embodiments, the GABA receptor conducts a phasic current. In some embodiments, the GABA receptor conducts a spontaneous inhibitory post-synaptic current (sIPSC).

In some embodiments, the GABA receptor receptor is on a solid-supported membrane (SSM). In some embodiments, a membrane fragment carrying the GABA receptor is adsorbed to a lipid monolayer painted over a functionalized electrode for electrophysiology recordings.

In some embodiments, the GABAergic current measured in the present method comprises a tonic current and/or a sIPSC. In some embodiments, a greater GABAergic current is indicated by (1) a larger average amplitude of the tonic current; (2) a higher average current density of the tonic current; (3) a larger average amplitude of the sIPSC; (4) a longer average decay time of the sIPSC; (5) or any combination of (1)-(4).

In some embodiments, the GABAergic current is measure by electrophysiological recording methods, including but not limited to whole-cell recording, perforated patch recording, single-unit recording, multi-unit recording, patch clamp recording, voltage-clamp recording, current-clamp recording, field potential measurement, amperometry measurement.

In some embodiments, the present method further comprises evaluating efficacy of a GABA receptor potentiator. In some embodiments, the greater increase in GABAergic current induced by the test agent, the more efficient the test agent acts as a GABA receptor potentiator. In some embodiments, the more sustained effect in GABAergic current induced by the test agent, the more efficient the test agent acts as a GABA receptor potentiator.

In some embodiments, the present method further comprises evaluating whether a GABA receptor potentiator is an allosteric modulator or a metabotropic modulator. In some embodiments, the method comprises measuring how long a GABAergic modulation effect can still be observed after the GABA receptor potentiator has been removed from the test system.

### Evaluating a Candidate GABAergic Modulator

In some embodiments, provided herein are also methods of determining whether a candidate GABAergic modulator is an allosteric modulator or a metabotropic modulator. In some embodiments, the evaluation is based on determining whether the modulation effect acutely rises immediately after a test system is exposed to the candidate GABAergic modulator. In some embodiments, the method comprises:

a) contacting a suitable test system with a candidate GABAergic modulator and spontaneously measuring a selected test parameter from the test system contacted with the candidate GABAergic modulator;

b) comparing the test parameter from the test system contacted with the candidate GABAergic modulator with the test parameter from the test system not contacted with the candidate GABAergic modulator.

In some embodiment, if the test parameter of the test system contacted with the candidate GABAergic modulator is different from the test parameter from the test system not contacted with the candidate GABAergic modulator, the candidate GABAergic modulator is an allosteric modulator.

In some embodiment, if the test parameter of the test system contacted with the candidate GABAergic modulator is not different from the test parameter from the test system not contacted with the candidate GABAergic modulator, the candidate GABAergic modulator is a metabotropic modulator.

In some embodiments, the evaluation is based on determining whether the modulation effect is sustained after the removal of the candidate GABAergic modulator from the test system. In some embodiments, the method comprises:

a) contacting a suitable test system with a candidate GABAergic modulator for a period of time sufficient for illicit a metabotropic effect;

b) removing the candidate GABAergic modulator from the test system;

c) measuring a test parameter from the test system contacted with the candidate GABAergic modulator;

d) comparing the test parameter from the test system contacted with the candidate GABAergic modulator with the test parameter from the test system not contacted with the candidate GABAergic modulator.

In some embodiments, if the test parameter of the test system contacted with the candidate GABAergic modulator is different from the test parameter from the test system not contacted with the candidate GABAergic modulator, the candidate GABAergic modulator is a metabotropic modulator.

In some embodiments, if the test parameter of the test system contacted with the candidate GABAergic modulator is not different from the test parameter from the test system not contacted with the candidate GABAergic modulator, the candidate GABAergic modulator is an allosteric modulator.

In some embodiments, the evaluation is based on determining whether the modulation effect is present when either an allosteric and modulation pathway or a metabotropic modulation pathway is blocked. In some embodiments, the method comprises:

a) block a allosteric modulation pathway in a suitable test system;

b) contacting the test system with a candidate GABAergic modulator;

c) measuring a test parameter from the test system contacted with the candidate GABAergic modulator;

d) comparing the test parameter from the test system contacted with the candidate GABAergic modulator with the test parameter from the test system not contacted with the candidate GABAergic modulator.

In some embodiments, if the test parameter of the test system contacted with the candidate GABAergic modulator is different from the test parameter from the test system not contacted with the candidate GABAergic modulator, the candidate GABAergic modulator is a metabotropic modulator.

In some embodiments, if the test parameter of the test system contacted with the candidate GABAergic modulator is not different from the test parameter from the test system not contacted with the candidate GABAergic modulator, the candidate GABAergic modulator is an allosteric modulator.

In some embodiments, blocking the allosteric pathway is achieved by inactivating the allosteric binding site of the GABA receptor. In some embodiments, blocking the allosteric pathway is achieved by mutate the GABA receptor at positions essential for allosteric interaction.

In some embodiments, the method comprises:

a) block a metabotropic modulation pathway in a suitable test system;

b) contacting the test system with a candidate GABAergic modulator;

c) measuring a test parameter from the test system contacted with the candidate GABAergic modulator;

d) comparing the test parameter from the test system contacted with the candidate GABAergic modulator with the test parameter from the test system not contacted with the candidate GABAergic modulator.

In some embodiments, if the test parameter of the test system contacted with the candidate GABAergic modulator is different from the test parameter from the test system not contacted with the candidate GABAergic modulator, the candidate GABAergic modulator is a allosteric modulator.

In some embodiments, if the test parameter of the test system contacted with the candidate GABAergic modulator is not different from the test parameter from the test system not contacted with the candidate GABAergic modulator, the candidate GABAergic modulator is an metabotropic modulator.

In some embodiments, blocking the metabotropic pathway is achieved by inactivating one or more signal transduction nodes in the metabotropic signaling pathway. For example, in some embodiments, inactivating the metabotropic signaling pathway is achieved by inhibiting a protein kinase in the signaling pathway (such as PKC), removing an effector molecule from the test system, such as the heterotrimeric G protein, or inactivating an effector molecule, such as inhibiting mPR.

### System for Screening

According to the present disclosure, the test system for identifying and evaluating GABAergic modulators can be either an in vitro system or an in vivo system. In some embodiments, the test system comprises a membrane having at least one membrane-associated GABA receptor. In some embodiments, the membrane having at least one membrane-associated GABA receptor is part of a cell membrane. In some embodiments, the cell membrane is a post-synaptic membrane. In some embodiments, the cell membrane is a pre-synaptic membrane. In various embodiments, the one or more GABA receptors are located in a synaptic area of the postsynaptic membrane, an extrasynaptic area of the postsynaptic membrane or both. In some embodiments, the membrane having at least one membrane-associated GABA receptor is part of a cell. In some embodiments, the membrane having at least one membrane-associated GABA receptor is an artificially constructed membrane having a solid support.

In some embodiments, the test system comprises a cell expressing at least one GABA receptor subunit. In some embodiments, the cell forms a synapse with at least one neighboring cell. In some embodiments, the at least one GABA receptor subunit, after expression, assembles into a GABA receptor that is inserted into a synaptic area or extrasynaptic area of the cell membrane. In some embodiments, the at least one GABA receptor subunit, upon expression, is assembled into a GABA_{A} receptor. In some embodiments, the at least one GABA receptor subunit, upon expression, is assembled into a synaptic GABA_{A} receptor. In some embodiments, the at least one GABA receptor subunit, upon expression, is assembled into an extrasynaptic GABA_{A} receptor. In some embodiments, the at least one GABA receptor subunit, upon expression, is assembled into a GABA_{B} receptor.

In some embodiments, the test system comprises a cell expressing at least one GABA receptor subunit selected from α(1-6), β(1-3), γ(1-3), γ(1-3), δ, ε(1-3), θ, and π subunits, or a functional domain thereof. In some embodiments, the test system comprises a cell expressing at least one GABA receptor subunit selected from α(1, 2, 3 or 5), β and γ subunits, or a functional domain thereof. In some embodiments, the test system comprises a cell expressing at least one GABA receptor subunit selected from α(4/6), β, δ subunits, or a functional domain thereof. In some embodiments, the test system comprises a cell expressing 2α, 2β and 1γ (or 1δ) GABA receptor subunits, or one or more functional domains thereof. In some embodiments, the test system comprises a cell expressing at least α1, β2 and γ2 GABA receptor subunits, or one or more functional domains thereof. In some embodiments, the test system comprises a cell expressing at least α4, β3 and δ GABA receptor subunits, or one or more functional domains thereof.

In some embodiments, the test system comprises a cell having one or more endogenous genes encoding for at least one GABA receptor subunit. In some embodiments, the test system comprises a cell having one or more artificial genetic constructs encoding for at least one GABA receptor subunit, or a functional domain thereof. In some embodiments, the test system comprises a cell that comprises one genetic construct encoding for multiple GABA receptor subunits or functional domains thereof, which coding sequences are operably connected to the same expression regulatory element. In some embodiments, the test system comprises a cell that comprises multiple genetic constructs, each encoding for one GABA receptor subunit, or a functional domain thereof, under the control of an expression regulatory element operably linked to the coding sequence.

In some embodiments, the cells expressing at least one GABA receptors are cultured from an established cell line. In some embodiments, the cells expressing at least one GABA receptors are isolated from a tissue sample. In some embodiments, the cells expressing at least one GABA receptors are isolated from a model animal bearing mutations in one or more GABA genes. In some embodiments, the model animal is a mouse having a S408/9A mutation in the β3 GABA subunit, a S443A mutation in the α4 GABA subunit or a combination thereof. In some embodiments, the effect of S408/9 phosphorylation is mimicked by mutation of these serine residues to alanine residues. Vien et al. 2015: PNAS 112: 14805-10.

Examples of cells or cell lines that can be used in connection with the present invention include but are not limited to a brain cell, a neuron, a dentate gyms granule cell (DGGC), a motor neuron, GT1-7 cell, a LTK cell, a CHO cell, a HEK293 cell, a human derived stem cells, a differentiated induced pluripotent stem cell (iPSC), a primary rodent cell (neurons, astrocytes, oligodendrocytes, spinal cord, microglia), an immune cell such as a Jurkat Cell, neuroblastoma cell lines such as SH-SY5Y, NTERA-2, PC12, IMR-32, breast cancer cell lines such as MDA-MB-231, MDA-MB-468, MCF-7, T47D, glial cell lines such as S42 Schwann, lung Cancer cell lines such as A549, and other cell lines such as HeLa, COS7, HEK 293T.

In some embodiments, the cells are located in a tissue or on a tissue surface, such as a muscle surface preparation, a brain slice preparation such as a hippocampal slice preparation, a spinal cord preparation, a reproductive tissue (e.g., ovaries preparation), and a pancreatic islet preparation. In some embodiments, the cells comprise a presynaptic membrane. In some embodiments, the cells comprise a postsynaptic membrane.

In some embodiments, the test system comprises a synapse comprising a presynaptic membrane, a post-synaptic membrane and a synaptic cleft. In some embodiments, the synapse is formed by a presynaptic cell and a postsynaptic cell. In some embodiments, the post-synaptic cell membrane comprises membrane-associated GABA receptors in the synaptic regions. In some embodiments, the post-synaptic cell membrane further comprises membrane-associated GABA receptors outside the synaptic regions. In some embodiments, the post-synaptic cell membrane further comprises membrane-associated GABA receptors both inside and outside the synaptic regions.

In some embodiments, the test system is a cell comprising a reporting mechanism that is indicative of the expression level of at least one GABA receptor subunit. For instance, in some embodiments, the test system comprises a cell transfected with a reporter gene capable of producing a detectable signal upon expression, the intensity of the signal indicative of the expression level of at least one GABA receptor subunit in the cell. In some embodiments, the expression of the reporter gene is regulated by a regulatory element of a GABA gene. For example, in various embodiments, the reporting gene may encode for a fluorescent protein or a luciferase protein under the control of a regulatory element from a GABA gene. In some embodiments, the test system is a cell comprising a reporting mechanism that is indicative of the phosphorylation status of at least one GABA receptor subunit.

In some embodiments, the test system is a cell-based system. In some embodiments, the cell-based system comprises one or more components that act as signal transduction nodes of a signaling pathway. In some embodiments, the cell-based system comprises one or more signal transduction nodes of a mPR-mediated signaling pathway. In some embodiments, the cell-based system comprises one or more components selected from: a mPR, a heterotrimeric G protein, a Gαi subunit of a a heterotrimeric G protein; a Gβγ subunit of a a heterotrimeric G protein, phospholipase C (PLC), phosphoinositide 3-kinase (PI3K), protein kinase C (PKC), diacylglycerol (DAG), inositol 1,4,5-trisphosphate (IP3), Ca2+/calmodulin-dependent protein kinase II (CaMKII) ATP, cAMP, and intracellular Ca²⁺, at least one GABA receptor subunit.

### EXAMPLES

### Example 1 Recombinant GABA_{A} Pharmacology

Cellular electrophysiology was used to measure the pharmacological properties of ALLO, ganaxolone and SGE-516 in heterologous cell systems. Compounds were tested for their ability to affect GABA mediated currents at a submaximal agonist dose (GABA EC₂₀=2 μM).

LTK cells were stably transfected with the α1β2γ2 subunits of the GABA receptor and CHO cells are transiently transfected with the α4β3γ subunits via the Lipofecatamine method. Cells were passaged at a confluence of about 50-80% and then seeded onto 35 mm sterile culture dishes containing 2 ml culture complete medium without antibiotics or antimycotics. Cells were cultivated at a density that enabled the recording of single cells without visible connections to other cells.

Whole-cell currents were measured with HEKA EPC-10 amplifiers using PatchMaster software. Bath solution for all experiments contained (in mM): NaCl 137, KCl 4, CaCl₂ 1.8, MgCl₂ 1, HEPES 10, D-Glucose 10, pH 7.4 with NaOH. Intracellular (pipette) solution contained (in mM): KCl 130, MgCl2 1, Mg-ATP 5, HEPES 10, EGTA 5, pH 7.2 with KOH. During experiments, cells and solutions were maintained at room temperature (19° C.-30° C.). For manual patchclamp recordings, cell culture dishes were placed on the dish holder of the microscope and continuously perfused (1 ml/min) with bath solution. After formation of a Gigaohm seal between the patch electrode and the cell (pipette resistance range: 2.5 MΩ-6.0MΩ; seal resistance range: >1 GΩ) the cell membrane across the pipette tip was ruptured to assure electrical access to the cell interior (whole-cell patch configuration).

Cells were voltage-clamped at a holding potential of −80 mV. GABA_{A} receptors were activated by 2 μM GABA and compounds were sequentially applied at increasing concentrations for 30 s prior to a 2 s application of GABA. GABA and compounds were applied to cells via the Dynaflow perfusion system (Cellectricon, Sweden). Test compounds were dissolved in DMSO to form 10 mM stock solution and serially diluted to 0.01, 0.1, 1, and 10 μM in bath solution. There was no effect on GABA currents when DMSO was applied to cells at its maximal concentration in solution (0.1%). All concentrations of test compound were tested on each cell. The relative percentage potentiation was defined as the peak amplitude in response to GABA EC20 in the presence of test compound divided by the peak amplitude in response to GABA EC20 alone, multiplied by 100.

### Hippocampal Slice Preparation

Brain slices were prepared from 3- to 5-week-old male C57 mice. Mice were anesthetized with isoflurane, decapitated, and brains were rapidly removed and submerged in ice-cold cutting solution containing (mM): 126 NaCl, 2.5 KCl, 0.5 CaCl₂, 2 MgCl₂, 26 NaHCO₃, 1.25 NaH₂PO₄, 10 glucose, 1.5 sodium pyruvate, and 3 kynurenic acid. Coronal 310 μm thick slices were cut with the vibratome VT1000S (Leica Microsystems, St Louis, Mo., USA). The slices were then transferred into incubation chamber filled with prewarmed (31-32° C.) oxygenated artificial cerebro-spinal fluid (ACSF) of the following composition (in mM): 126 NaCl, 2.5 KCl, 2 CaCl₂, 2 MgCl₂, 26 NaHCO₃, 1.25 NaH₂PO₄, 10 glucose, 1.5 sodium pyruvate, 1 glutamine, 3 kynurenic acid and 0.005 GABA bubbled with 95% O₂-5% CO₂. Slices were allowed to recover at 32° C. for at least 1 hr before recording. Exogenous GABA was added in an attempt to standardize ambient GABA in the slice and provide an agonist source for newly inserted extrasynaptic GABA_{A}Rs.

### Example 2 Electrophysiology Recordings

After recovery, a single slice was transferred to a submerged, dual perfusion recording chamber (Warner Instruments, Hamden, Conn., USA) on the stage of an upright microscope (Nikon FN-1) with a 40× water immersion objective equipped with DIC/IR optics. Slices were maintained at 32° C. and gravity-superfused with ACSF solution throughout experimentation and perfused at rate of 2 ml/min with oxygenated (O₂/CO₂ 95/5%) ACSF.

Whole-cell currents were recorded from the dentate gyms granule cells (DGGCs) in 310-μm-thick coronal hippocampal slices. Patch pipettes (5-7MΩ) were pulled from borosilicate glass (World Precision Instruments) and filled with intracellular solution of the composition (in mM) as follows: 140 CsCl, 1 MgCl₂, 0.1 EGTA, 10 HEPES, 2 Mg-ATP, 4 NaCl and 0.3 Na-GTP (pH=7.2 with CsOH). A 5 min period for stabilization after obtaining the whole-cell recording conformation (holding potential of −60 mV) was allowed before currents were recorded using an Axopatch 200B amplifier (Molecular Devices), low-pass filtered at 2 kHz, digitized at 20 kHz (Digidata 1440A; Molecular Devices), and stored for offline analysis.

### Example 3 Electrophysiology Analysis

For tonic current measurements, an all-points histogram was plotted for a 10 s period before and during 100 μM picrotoxin application, once the response reached a plateau level. Recordings with unstable baselines were discarded. Fitting the histogram with a Gaussian distribution gave the mean baseline current amplitude and the difference between the amplitudes before and during picrotoxin was considered to be the tonic current. The negative section of the all-points histogram which corresponds to the inward IPSCs was not fitted with a Gaussian distribution (Kretschmannova et al., 2013; Nusser and Mody, 2002). Series resistance and whole-cell capacitance were continually monitored and compensated throughout the course of the experiment. Recordings were eliminated from data analysis if series resistance increased by >20%. Spontaneous inhibitory post-synaptic currents (sIPSCs) were analyzed using the mini-analysis software (version 5.6.4; Synaptosoft, Decatur, Ga.). Minimum threshold detection was set to 3 times the value of baseline noise signal. To assess sIPSC kinetics, the recording trace was visually inspected and only events with a stable baseline, sharp rising phase, and single peak were used to negate artifacts due to event summation. Only recordings with a minimum of 200 events fitting these criteria were analyzed. sIPSCs amplitude, and frequency from each experimental condition was pooled and expressed as mean±SEM. To measure sIPSC decay, 100 consecutive events were averaged and the decay was fitted to a double exponential and the weighted decay constant (tw) was determined. Statistical analysis was performed by using Student t-test (paired and unpaired where appropriate), where p<0.05 is considered significant.

### Example 4 Metabolic Labeling and Biotinylation

Hippocampi were dissected out of acute slices from 8 to 12 week old C57/B16 mice and lysed with phosphate buffer including: 20 mM Tris-HCl, 150 mM NaCl, 5 mM EDTA, 10 mM NaF, 2 mM Na3VO4, 10 mM pyrophosphate, 0.1% SDS and 1% Triton after drug treatment. The β3 subunit was isolated using immunoprecipitation with β3 antibodies, after correction for protein content and the specific activity of labeling. Results were attained by SDS/PAGE followed by autoradiography (Abramian et al., 2010). For biotinylation experiments hippocampi were dissected out of acute slices from 8 to 12 week old C57/B16 mice and incubated in artificial cerebrospinal fluid (ACSF) described above at 30° C. for 1 h for recovery before experimentation. Slices were then placed on ice and incubated for 30 min with 1 mg/mL NHS-SS-biotin (Pierce). Excess biotin was removed by a 50 mM glycine quenching buffer, followed by washing of slices three times in ice-cold ACSF. The tissue was snap frozen on dry ice for 5 min, thawed at 4° C. and lysed. The lysates were solubilized with 2% Triton at 4° C. on a rotating wheel for 1 h. The insoluble material was removed by centrifugation, and 350-500 μg of protein lysate were incubated with NeutrAvidin beads (Pierce) for 18-24 h at 4° C. Bound material was eluted with sample buffer and subjected to SDS/PAGE and then immunoblotted with the indicated antibodies. Blots were then quantified using the CCD-based ChemiDoc XRS [symbol] system. Antibodies against the β3 subunit and the phospho-β3 antibody were generated and verified by the laboratory of S.J.M. (Vien et al., 2015).

### Example 5: Comparing the Effects of Endogenous and Synthetic Neurosteroids on the Activity of Recombinant GABA_{A}Rs

ALLO and ganaxolone are known PAMs of both synaptic and extrasynaptic GABA_{A}R-mediated currents. The ability of candidate compound SGE-516 to act as a PAM was compared to ALLO and ganaxolone using the whole-cell recordings of recombinant human GABAA receptors expressed in mammalian cells. The α1β2γ2 or α4β3δ subunit combinations were chosen as representatives of typical synaptic and extrasynaptic GABAA receptors respectively. Similar to previous reports (Botella et al., 2015), ALLO, ganaxolone and SGE-516 allosterically potentiated currents induced by EC₂₀ concentration of GABA in a concentration-dependent manner in both synaptic- and extrasynaptic-type GABA_{A}Rs. ALLO potentiated α1β2γ2 receptors with an EC₅₀ of 115 nM and Eₘₐₓ of 229% and potentiated α4β3δ receptors with an EC₅₀ of 57 nM and Eₘₐₓ of 426%. SGE-516 potentiated α1β2γ2 receptors with an EC₅₀ of 61 nM and Emax of 219%. Likewise, SGE-516 potentiated α4β3δ receptors with an EC₅₀ of 193 nM and Emax of 400%. Ganaxolone potentiated α1β2γ2 receptors with an EC₅₀ of 256 nM and Eₘₐₓ of 307% and potentiated α4β3δ receptors with an EC₅₀ of 94 nM and Eₘₐₓ of 225% (Table 1).

To confirm the presence of the γ2 subunit in α1β2γ2 receptors, the effects of diazepam (a benzodiazepine PAM specifically for synaptic GABA_{A}Rs) was evaluated (data not shown). It was observed that diazepam potentiates the GABA evoked currents in α1β2γ2 receptors with an EC₅₀ of 69 nM and Eₘₐₓ of 184%. In contrast, it did not alter GABA evoked currents in α4β3δ receptors. The results indicate that like ALLO, and ganaxolone, SGE-516 is a potent and efficacious PAM for both synaptic and extrasynaptic type GABA_{A}Rs.

TABLE 1
Properties of neuroactive steroids on recombinant GABA_{A}Rs.

| | α1β2γ2 | | α4β3δ | |
|---|---|---|---|---|
| Compound | EC₅₀ (pEC₅₀ [symbol] S.E.M.) | Eₘₐₓ [symbol] S.E.M. (%) | EC₅₀ (pEC₅₀ [symbol] S.E.M.) | Eₘₐₓ [symbol] S.E.M. (%) |
| ALLO | 115 (6.9 ± 0.2) | 229 ± 19 | 57 (7.2 ± 0.3) | 426 ± 42 |
| SGE-516 | 61 (7.2 ± 0.3) | 219 ± 21 | 193 (6.7 ± 0.1) | 505 ± 19 |
| Ganaxolone | 256 (6.8 ± 0.2) | 400 ± 27 | 94 (7.0 ± 0.1) | 225 ± 8 |

EC₅₀ values are given in nM with [symbol] pEC₅₀ ± S.E.M., n = 3 for all. [symbol]
indicates data missing or illegible when filed

### Example 6: Acute Exposure to NASs Allosterically Potentiates Tonic Current in the Dentate Gyms Granule Cells

The results from the above experiments with synaptic-, and extrasynaptic-like GABA_{A}Rs expressed in HEK293 cells demonstrated the ability of ALLO, SGE-516, and ganaxolone to potentiate sub-maximal GABA-mediated currents. The ability of acute application of these NASs to allosterically modulate phasic and tonic currents in dentate gyms granule cells (DGGCs) in hippocampal slices from 3 to 5 weeks old male C57/B16 mice was examined. Hippocampal slices from p21-35 (C57/B16) mice were allowed to recover for at least 1 h following slicing. Slices were transferred to the recording chamber of the icroscope. After achieving the whole-cell configuration approximately 10 min was allowed for membrane currents to stabilize. Hippocampal slices were acutely exposed to 100 nM ALLO, SGE-516, or ganaxolone (Ganax) for 10 min followed by 100 μM picrotoxin (PTX).

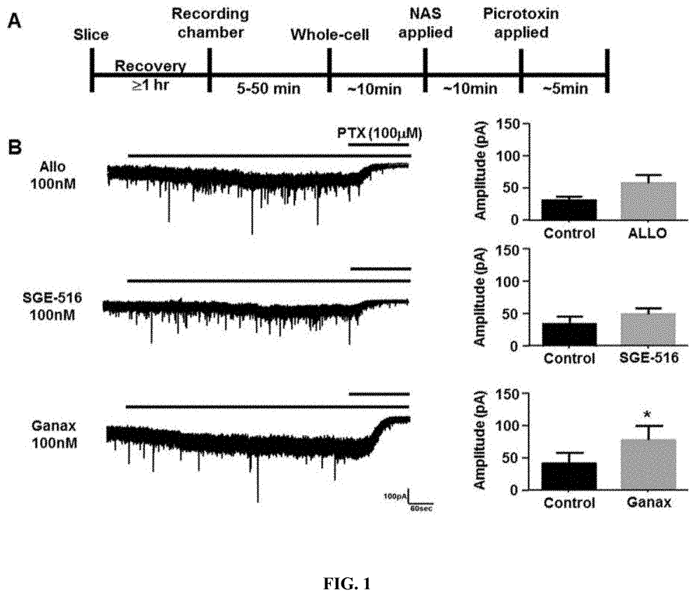

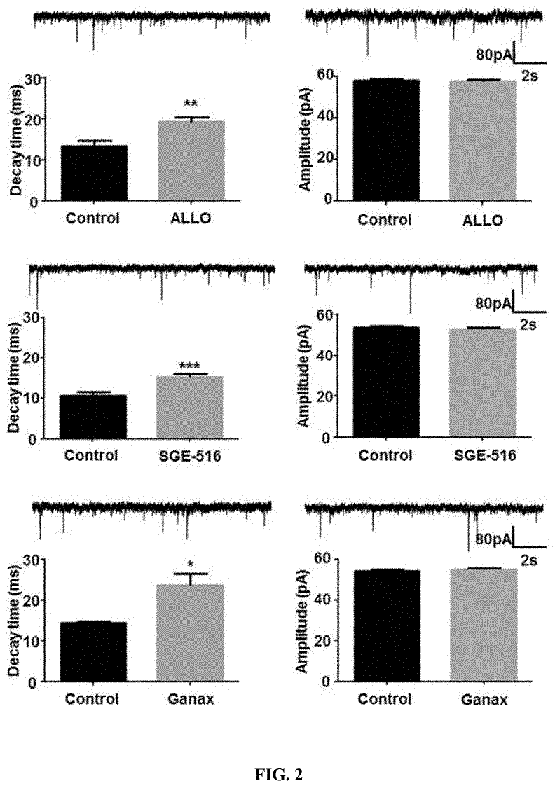

Both ALLO, and SGE-516 modulated the tonic holding current in DGGCs in hippocampal slices (FIG. 1). At 100 nM, ALLO modulated the tonic current from 31.8±5.9 pA, to 58.3±13.0 pA, (n=5) and 100 nM SGE-516 modulated the tonic current from 34.2±11.1 pA, to 49.2±8.9 (n=7). The only significant modulation was observed with 100 nM ganaxolone which modulated DGGC tonic current from 42.1±17.5 to 78.8±23.2 pA (n=6; p=0.004 paired t-test).

### Example 7: Comparing the Acute Effects of NASs on Phasic Currents in DGGCs

The properties of inhibitory synaptic currents in DGGCs before and during exposure to NASs (FIG. 2) were compared. It was observed that there was no significant difference in the mean sIPSC amplitude before and during exposure with 100 nMALLO (p=0.85, n=5), 100 nM SGE-516 (p=0.46, n=7) and 100 nM ganaxolone (p=0.07, n=6) (Table 2). However, the mean sIPSC decay time significantly increased in the presence of ALLO, SGE-516, and ganaxolone (p=0.03, p=0.01, and p=0.04 respectively, Table 2).

TABLE 2
Allosteric modulation of DGGC sIPSCs evoked by 100 nM ALLO, SGE-516 and ganaxolone.

| sIPSC | Control | ALLO | Control | SGE-516 | Control | Ganax |
|---|---|---|---|---|---|---|
| Amplitude (pA) | 59.2 ± 0.7 | 59.1 ± 0.6 | 53.7 ± 0.7 | 52.9 ± 0.7 | 54.2 ± 0.6 | 55.1 ± 0.7 |
| Frequency (Hz) | 4.5 ± 0.9 | 3.4 ± 1.0 | 4.1 ± 0.4 | 4.6 ± 0.6 | 6.4 ± 1.3 | 5.6 ± 1.7 |
| Decay (ms) | 13.4 ± 1.3 | 19.2 ± 1.3^{a} | 10.5 ± 1.1 | 15.1 ± 0.8^{b} | 14.4 ± 0.5 | 23.6 ± 2.9^{c} |

Data is mean SEM. n = 5 to 7 neurons.
^{a}p = 0.01.
^{b}p = 0.03.
^{c}p = 0.04.

### Example 8: Exposure to NASs Metabotropically Enhance Tonic Current in DGGCs

In addition to the allosteric modulation of GABA_{A} receptors, THDOC, exerts sustained effects on GABAergic tonic current by enhancing the PKC-dependent phosphorylation of the α4 and β3 subunits, leading to enhanced insertion and stability of GABA_{A}Rs into the membrane and a long lasting increase in tonic current (Abramian et al., 2010, 2014).

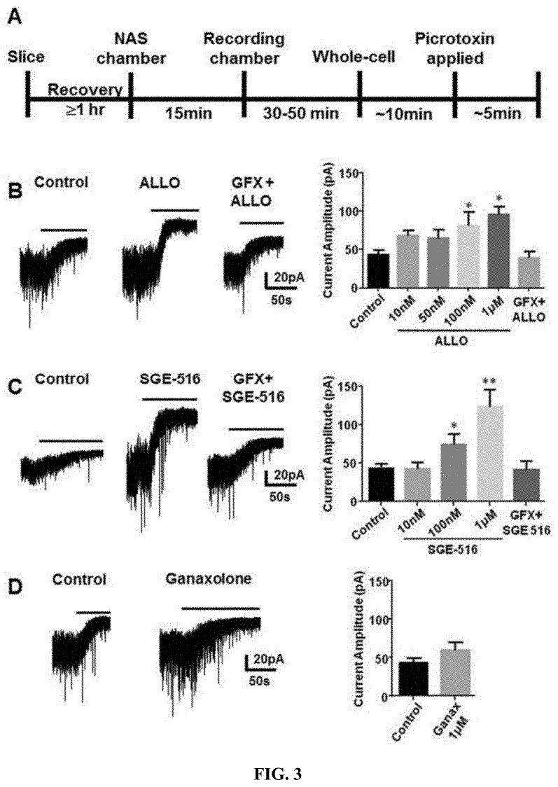

The sustained effects of ALLO, or the new synthetic NAS SGE-516 on the tonic current in DGGCs in hippocampal slices from 3 to 5 week old C57 male mice was analyzed. Hippocampal slices were allowed to recover for at least 1 h following slicing. Slices were then incubated for 15 min in a chamber containing NASs dissolved in ACSF. Slices were then transferred to the recording chamber of the microscope followed by a wash period between 30 and 60 min of continuous perfusion of NAS-free ACSF before recordings were started. Recordings were made from DGGCS in hippocampal slices from p21-35 C57 mice in the presence of 5 μM GABA followed by 100 μM picrotoxin and the difference in holding current was then determined. (FIG. 3A).

Slices exposed to ALLO, or SGE-516 demonstrated a concentration-dependent increase in the tonic current measured by the addition of picrotoxin with the maximal effects at 1 μM. Control, vehicle-treated slices had a tonic current of 43.9±5.7 pA (n=12), whereas the tonic currents for slices treated with SGE-516 (1 μM) was 123.0±22.2 pA, (n=6, p=0.0003), or ALLO (1 μM) was 95.8±10.8 pA (n=4, p=0.0005, FIG. 3B&C).

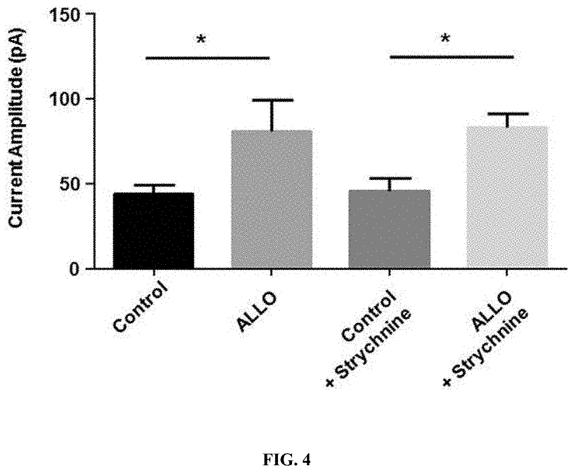

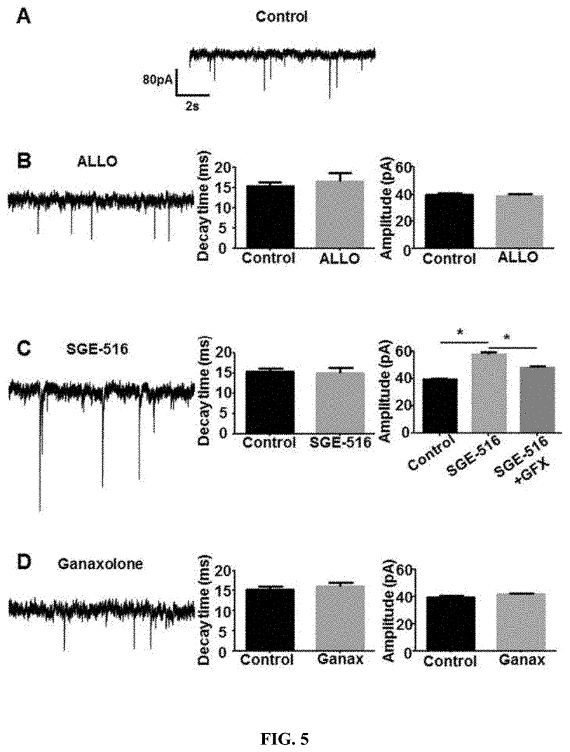

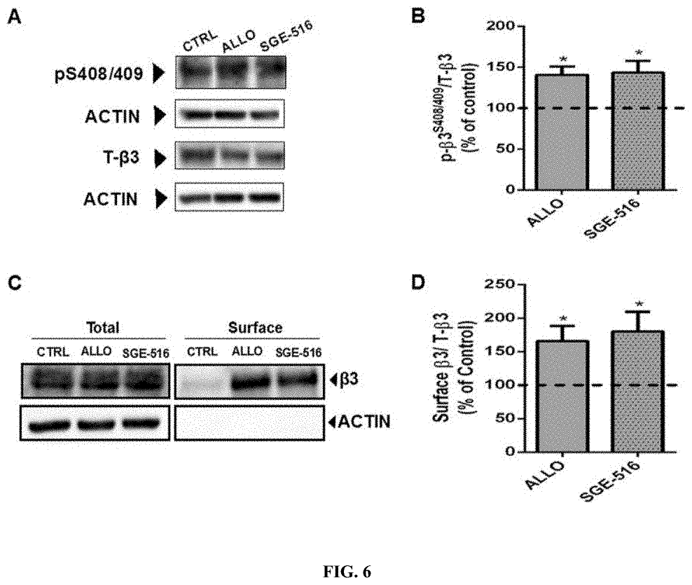

In addition, the metabotropic effect following 15 min incubation with synthetic NAS, ganaxolone was also examined. In contrast to the naturally occurring NASs THDOC, and ALLO, and the synthetic NAS, SGE-516, ganaxolone (1 μM) did not significantly alter the magnitude of tonic current in DGGCs (57.4±6.3 pA, n=7, p=0.14, FIG. 3D). To assess if the effects of NASs are dependent upon PKC, hippocampal slices were treated with the established PKC inhibitor GF 109203X (GFX 50 μM) for 15 min followed by co-exposure of to ALLO, or SGE-516 and GFX for 15 min. When tonic current was measured following ≤30 min washout, there was no significant difference to the tonic current measured in ALLO/GFX, or SGE-516/GFX treated slices with vehicle treated slices (FIG. 3). The tonic current was measured by blocking extrasynaptic GABA_{A}Rs with picrotoxin. Because picrotoxin also inhibits glycine receptors, the contribution of glycine receptors to tonic current was examined by using the specific glycine receptor inhibitor, strychnine. There was no difference in tonic current measured under control conditions in the absence or presence of strychnine (100 nM). Similarly, there was no difference in the increase in tonic current following a 15 min exposure to 100 nM ALLO in the absence or presence of strychnine (FIG. 4). These results suggest that glycine receptors have an undetected contribution to tonic current in DGGCs and that the metabotropic increase in tonic current by NAS exposure do not involve glycine receptors.

Collectively, these results suggest that the exposure of hippocampal slices to SGE-516 and ALLO has a strong sustained metabotropic effects on tonic current. In contrast, ganaxolone produce major effects via allosteric mechanism as compared to metabotropic effects.

### Example 9 Neurosteroids Increase Phosphorylation of GABA_{A}Rs and Their Cell Surface Stability

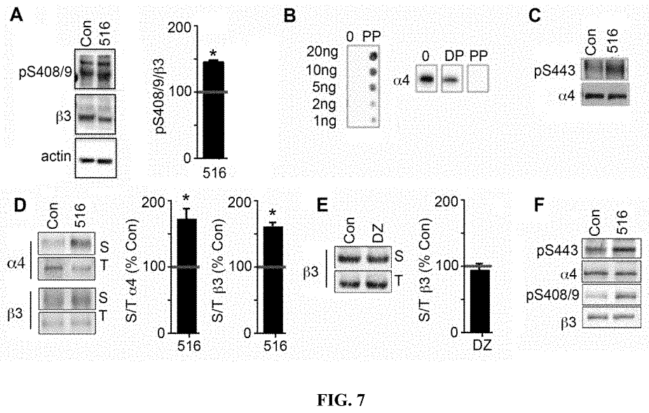

Treatment of hippocampal slices with SGE-516 increased the phosphorylation of S408/9 measured using pS408/9 antibodies. To produce antibodies specific for phosphorylated S443, rabbits were injected with a synthetic peptide corresponding to residues 336-447 of the murine α4 subunit in which the serine residue corresponding to S443 was chemically phosphorylated; PGSLGSASTRPA. Hippocampal slices were treated with vehicle (Con), or SGE-516 for 20 min. Slices were immunoblotted with pS408/9, β3, or actin antibodies. The ratio of pS408/9/β3 immunoreactivity was then normalized to control slices (100%=the line). The resulting antiserum exhibited high titer against the immunogen (FIG. 7A) and was accordingly subjected to affinity purification.

Affinity purified pS443 was used to immunoblot varying concentrations of the immunizing phosphor-peptide (PP). pS443 was used to immunoblot extracts of hippocampal slices treated without preadsorption (0), preadsorbed with the dephosphorylated (DP), or phosphorylated antigen (PP). Immunoblotting hippocampal extracts with pS443 revealed the presence of a major band of 64 kDa, identical in migration to the a4 subunit. Moreover, the detection of this band was blocked by the phospho- but not the dephospho-antigen (FIG. 7B).

Hippocampal slices were treated with vehicle (Con) or 100 SGE-516 for 5 min and then immunoblotted with pS443 and α4 antibodies as indicated. pS443 immunoreactivity in hippocampal slices was increased by exposure to SGE-516, while total α4 levels were unaffected (FIG. 7C).

Hippocampal slices were treated as outlined above. Treated slices were then subject to biotinylation and lysis, and surface fractions were isolated on immobilized avidin. Surface (S) and total (T) fractions were immunoblotted with α4 and β3 subunit antibodies. The results showed that SGE-516 increased the plasma membrane accumulation of both the α4 and β3 subunits (FIG. 7D).

Slices were treated with 100 nM diazepam (DZ) and its effect on cell surface stability of the β3 subunit was determined as outlined above. Diazepam (DZ) did not affect cell surface stability of the β3 subunit (FIG. 7E).

IP injection of SGE-516 also increased phosphorylation of S443 and S408/9 in the brains of mice sacrificed by focused microwave irradiation (FIG. 7F). C57/B16 mice injected with SGE-516 (5 mg/kg IP), or vehicle. 30 min after the treatment mice were sacrificed by microwave irradiation. SDS-soluble hippocampal extracts were then immunoblotted with pS443, α4, pS408/9, or β3 subunit antibodies.

### Example 10: Mutation of S408/9 in the β3 Blocks the Ability of SGE-516 to Induce Sustained Effects on GABAergic Inhibition

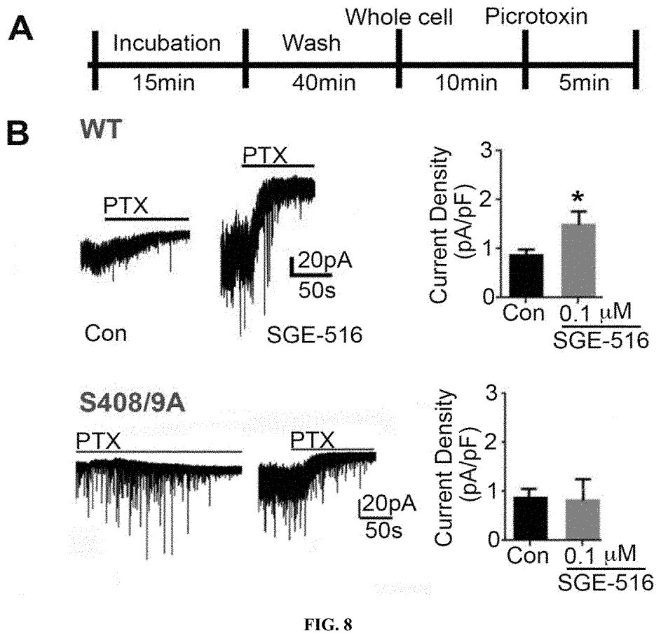

Consistent with the examples above, incubation of WT slices with 100 nM SGE-516 was followed by an extensive washout period. This treatment significant increased tonic current in DGGCs (FIG. 8A). In contrast, incubation of slices from S408/9A mice with SGE-516 under the same conditions did not modify tonic current in S408/9A mice (FIG. 8B).

### Example 11: Mutation of S408/9 in the β3 Blocks the Effects of SGE-516 on the Cell Surface Levels og GABA_{A}Rs

Consistent with the examples above, hippocampal slices from WT and S408/9A mice were treated for 20 min with 100 nM SGE-516 or vehicle (Con) and subjected to biotinylation followed by immunoblotting with β3 and α4 subunit antibodies. The ratio of surface/total (S/T) immunoreactivity was then normalized to levels seen in control (100%).

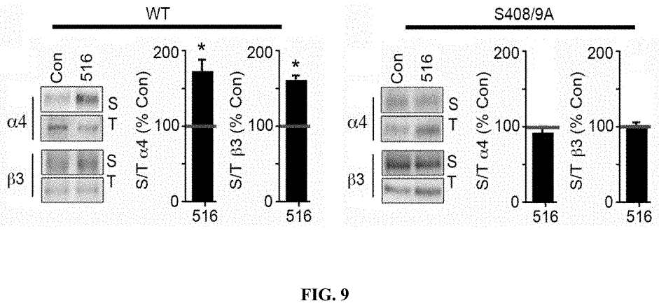

SGE-516 significantly increased the plasma membrane levels of GABA_{A}Rs containing the α4 and β3 subunits to 175-185% in hippocampal slices from WT mice (FIG. 9A). However, this effect was not seen in slices prepared from S408/9A mice (FIG. 9B).

### Example 12: Mutation of S408/9 in the β3 Subunit Does Not Block the Ability on Neurosteroids to Allosterically Modulate mIPSCs

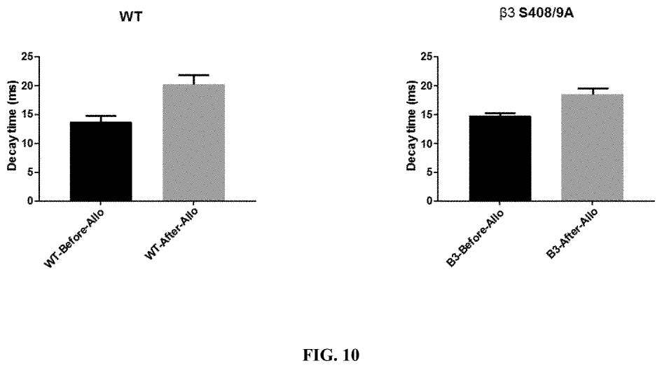

The ability of 100 nM ALLO to modulate the decay time of sIPSCs was compared in DGGCs from WT and S940A mice as detailed in FIG. 2. In contrast to WT, ALLO did not increase decay time in the mutant mice (FIG. 10).

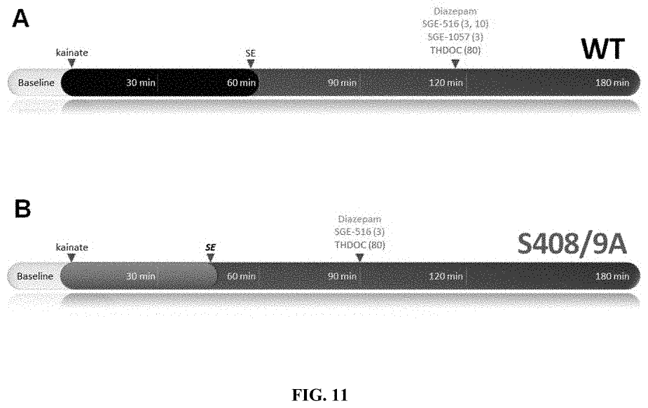

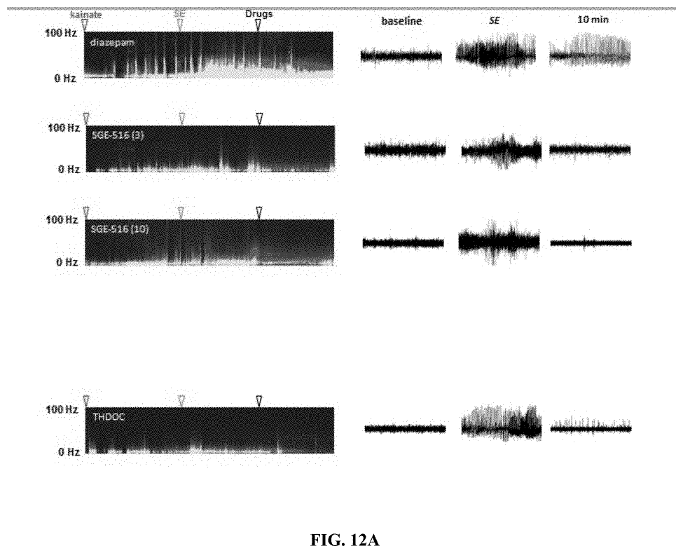

FIG. 11 shows the diagrams representing the protocols used to induced pharmacoresistant seizures in WT and S408/9A mice using kainite acid as measured using EEG recording. The time points used to test the anticonvulsant efficacy of benzodiazepines and neuroactive steroids to terminate seizure activity are also shown.

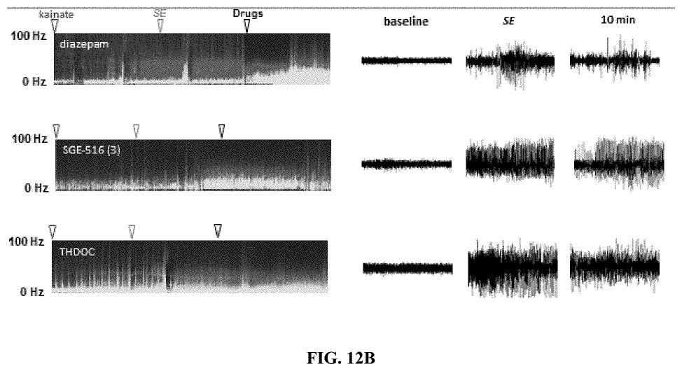

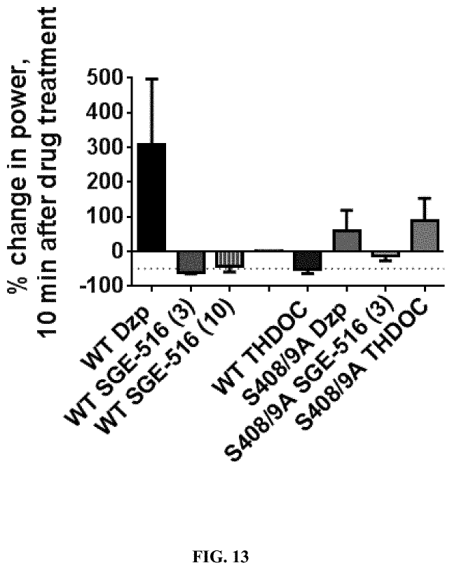

EEG power spectra are shown from WT mice undergoing SE induced by kainite >60 min (“SE” arrow), and EE. The ability of diazepam (10 mg/kg) SGE-516 (3 and 10 mg/kg) or THDOC (50 mg/kg). Representative EEG traces are shown at baseline, 60 after entrance into SE and 10 min after drug exposure. All drugs were injected IP as indicated by the “drug” arrow (FIG. 12A). The ability of diazepam, SGE-516 and THDOC to modify seizure activity in S408/9A mice was determined as detailed above (FIG. 12B). Seizure power was compared 10 min after exposure to the respective drugs. The only treatments that exhibited ≤50% reduction in power 10 minutes after treatment are SGE-517 (3 mg/kg) and THDOC (80 kg/mg) in wild type mice (FIG. 13).

### Example 13: Diversity in Ability of Neuroactive Steroids GABA PAM to Traffic GABA_{A} Receptors

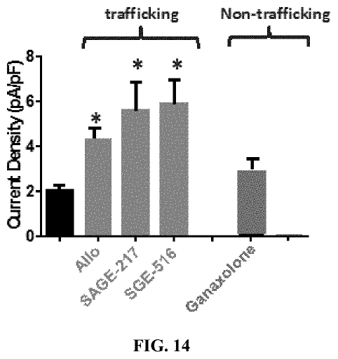

As shown in above examples, not all neurosteroids can modulate GABA receptor trafficking. As shown in FIG. 14, measured by the GABAergic current density, some neurosteroids, such as ALLO, SAGE-217, and SGE-516 modulated GABA receptor trafficking, while other neurosteroids, such as Ganaxolone did not modulate GABA receptor trafficking.

### Example 14: ALLO and P4 Increase S408/9 Phosphorylation in GTI-7 Cells

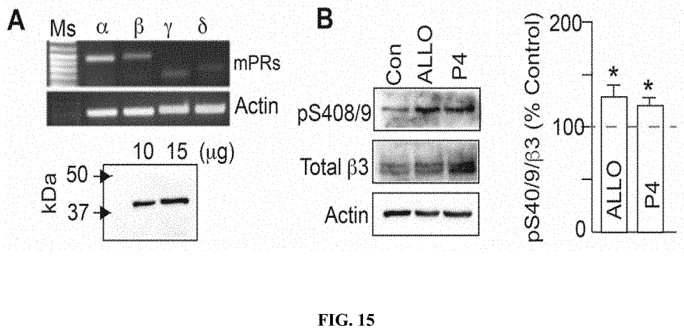

GT1-7 cells that co-express mPRα and GABA_{A}Rs were used to measure the level of phosphorylation at S408/9 position of β3 subunit. Quantitative PCR analysis indicated the enrichment of the mPRα mRNA in GT1-7 cells (FIG. 15A upper, figure taken from Thomas and Pang 2012). Expression of mPRα was confirmed using western blotting. 10 and 15 μg of SDS-soluble extracts from GT1-7 cells were immunoblotted with an mPRα specific antibody (FIG. 15A lower).

GT1-7 cells were treated with 100 nM AllO or P4 for 15 min and immunoblotted with antibodies specifically recognizing β3, actin, and phosphonate β3 subunit (pS408/9). The ratio of pS408/9 β3 subunit immunoreactivity were then normalized to levels seen in vehicle treated to controls, n=6. As shown in FIG. 15B, ALLO and P4 increase S408/9 phosphorylation in GT1-7 cells.

### Example 15: Internal ALLO and ORG Induce Sustained Increases in GABA-Evoked Currents Recorded from GTI-7 Cells

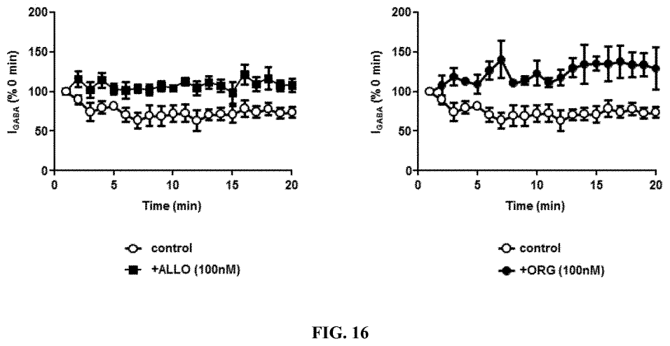

Patch-clamp recordings were made from GT1-7 cells. The magnitude of the GABA-induced currents (I_{GABA}) at EC20 agonists concentration were then measured, in the presence of control electrolyte or that supplemented with 100 nM ALLO, or 100 nM ORG OD 02-0 over a time course of 20 min. As shown in FIG. 16, ALLO and ORG OD 02-0 induced sustained increases in GABA-evoked currents recorded from GT1-7 cells.

### Example 16: ORG Does Not Acutely Modulate of the Function of GABA_{A}Rs Composed of α4β3 Subunits

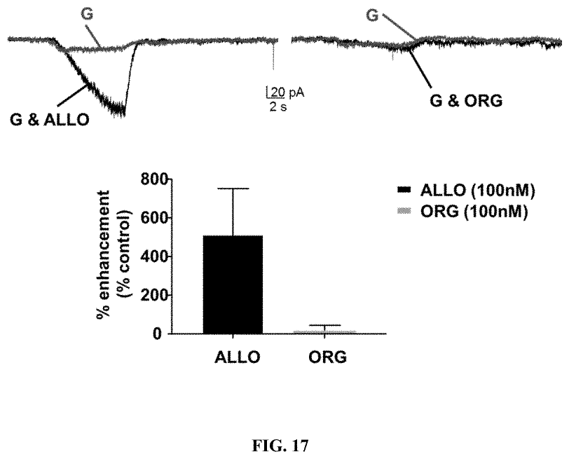

The magnitude of GABA-induced currents (I_{GABA}) was measured in HEK-293 expressing GABA_{A}Rs composed of α1 and β3 subunits. The effects of rapidly applied GABA (G), GABA and 100 nM ALLO (G&ALLO), or GABA and 100 mM ORG-020 (G&ORG) on GABA-evoked current (I_{GABA}) was then determined. Sample traces are shown in FIG. 17 upper panel. This data was then use to determine the potentiation of the GABA current by both drugs. As shown in FIG. 17 lower panel, ORG OD 02-0 compound did not acutely modulate of the function of GABA_{A}Rs composed of α4β3 subunits.

### Example 17: P4 and ORG-020 Regulates S408/9 Phosphorylation in Hippocampal Slices

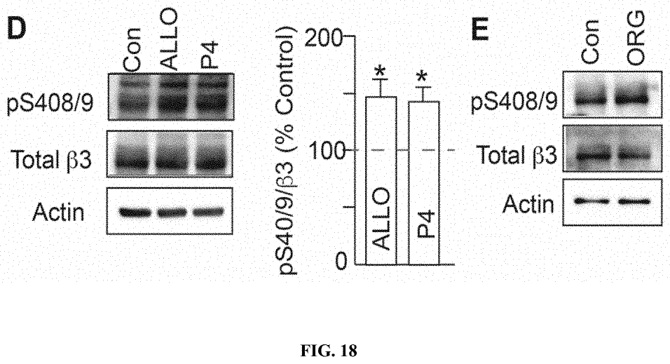

Hippocampal slices were treated with 100 nM ALLO or P4 (progesterone), and S408/9 phosphorylation was then determined as detailed above, n=4 slices (FIG. 18D). Hippocampal slices were treated with 100 nM ORG (FIG. 18E). S408/9 phosphorylation was examined using immunoblotting. In all panels; *=significantly different to control p<0.05 (one way ANOVA with Dunnet's multiple comparisons post-hoc test). As shown, P4 and ORG OD 02-0 regulated S408/9 phosphorylation in hippocampal slices.

### Example 18: ORG-02-0 and P4 Regulate a Tonic Current in Hippocampal Slices

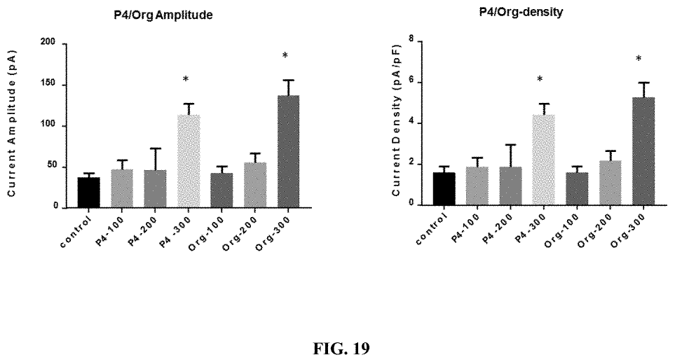

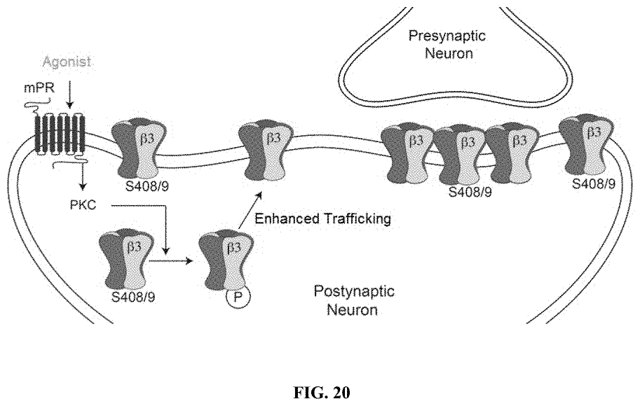

Dosage-dependent effect of P4 and ORG OD 02-0 compound in modulating GABAergic tonic current was measured. The ability of varying doses of P4 and ORG OD 02-0 to potentiate tonic current was determined as outlined in FIG. 3. The effects on current amplitude and density were then determined. *=significantly different to control p<0.05 (t-test n=6-8 cells). As shown in FIG. 19, both compounds exhibited dosage-dependent effect in modulating both the amplitude and density of GABAergic tonic current.

### Example 19: Development of a Membrane Progesterone Receptor Functional cAMP Assay using NTERA-2 Cells

### Overview

Certain mPRs activate Gs proteins, in turn stimulating adenylate cyclases (AC) and thus increasing the cellular cAMP level, while other mPRs activate Gi proteins, in turn inhibiting AC activity and thus decreasing the cellular cAMP level. mPR stimulation in NTERA-2 cells may thus yield an increase or a decrease of the cellular cAMP level, or both effects counteracting each other at the same time.

The cellular cAMP concentration of the NTERA-2 cells following incubation with the test compounds (reference mPR agonist: Progesterone) vs. buffer control is measured by HTRF as in the standard cAMP assays. To monitor a potential cAMP decrease through Gi activation, the AC activity is stimulated by addition of Forskolin.

To clarify the involvement of mPR-stimulated Gs vs. Gi protein activation in the net cAMP modulation (increase or decrease), the cells are pretreated with CTX (blocking Gs activation) vs. PTX (blocking Gi activation), respectively.

Based upon the intermediate results, one assay format measuring either cAMP increase or cAMP decrease (in presence of the AC activator Forskolin), respectively, is then selected for the further assay development.

### General Method of the cAMP Assay

The assay is performed in 96 well half area plates, in a total reaction volume of 20 μl per well. The cells are seeded the day before the assay (D-1 format), then starved overnight. The day of the assay, the cells are incubated in HBSS supplemented with 20 mM HEPES (pH 7.4) and 500 μM IBMX (phosphodiesterase inhibitor) in absence vs. presence of test compounds. Following the incubation, the cells are lysed and the cAMP is measured by HTRF (CisBio ref. 62AM2PEC).

### Phase 1: Feasibility and Determination of Optimal Cell Seeding Density

The day before the assay, NTERA-2 cells are seeded at different cell densities, then starved overnight.

8 different cell seeding densities are tested: 10 000/20 000/30 000/40 000/50 000/60 000/80 000 and 100 000 cells/well.

After overnight starvation, the cells are incubated with:
Buffer (basal level)
Progesterone (1 μM)
Org OD 02-0 (1 μM)
Forskolin (10 μM)
Forskolin (10 μM)+Progesterone (1 μM)
Forskolin (10 μM)+Org OD 02-0 (1 μM)

After 15 minutes incubation at room temperature, cAMP is measured by HTRF (CisBio ref. 62AM2PEC). One experiment (N=1) is performed in duplicate (n=2). Based upon the results, one optimal cell seeding density is selected for the subsequent experiments.

### Phase 2: CTX/PTX Pretreatment

The day before the assay, the cells are seeded at the density selected in the previous phase, then starved overnight, and treated overnight with:
CTX (20 μg/ml)
PTX (100 ng/ml)
CTX+PTX
No treatment

After the overnight starvation and CTX/PTX pre-treatment, the cells are incubated with:
Buffer (basal level)
Progesterone (1 μM)
Org OD 02-0 (1 μM)
Forskolin (10 μM)
Forskolin (10 μM)+Progesterone (1 μM)
Forskolin (10 μM)+Org OD 02-0 (1 μM)

After 15 minutes incubation at room temperature, cAMP is measured by HTRF (CisBio ref. 62AM2PEC).

One experiment (N=1) is performed in duplicate (n=2). Based upon the results, one condition (with or without Forskolin, CTX and PTX, respectively) is selected for the subsequent experiments.

### Phase 3: Assay Development and Compound Testing

The NTERA-2 cells are seeded as determined before, then stimulated as described/determined before, except for the following parameters:
6 different incubation times are tested: 5/10/15/20/30/45 minutes. One optimal incubation time will be selected.
2 different incubation temperatures are tested: 22/37° C. One optimal incubation temperature will be selected.
If applicable, different Forskolin concentrations are tested. One optimal incubation Forskolin concentration is selected (if applicable).
Test of 4 different DMSO concentrations: 0/0.1/0.3/1/3% final. The maximal DMSO concentration tolerated in the assay is determined.

Using the assay parameters optimized and selected throughout the different experiments, test compounds are then tested at each 8 concentrations. Results are expressed as percent of the control response compared to maximal effect obtained with the reference agonist Progesterone. The reference agonist Progesterone is be tested at several different concentrations to generate a concentration-response curve from which its EC50 value is calculated. Each one experiment (N=1) is performed in duplicate (n=2).

### Example 20: Development of a Membrane Progesterone Receptor Radioligand Binding Assay Using NTERA-2 Cells

### Overview

The standard (nuclear) progesterone receptor (PR) radioligand binding assay (described below) will be used as a starting point for the development of the mPR binding assay. Assay parameters such as the NTERA-2 cell membrane quantity, [³H]Progesterone concentration and incubation time and temperature are optimized. Finally, test compounds are tested in a competition binding assay.

### Phase 1: Cell Line Amplification and Membrane Preparation

NTERA-2 cells (ATCC ref. CRL-1973) are thawed and amplified according to the supplier's recommendations. Membrane homogenates are prepared according to standard protocol. The protein concentration is determined by Bradford assay.

### Phase 2: Feasibility and Determination of Optimal Protein Quantity

7 different NTERA-2 cell membrane protein quantities are tested: 10/25/50/75/100/150/200 μg/assay (negative control: without membranes).

The membranes will be incubated with 2 nM [³H]Progesterone (±1 μM unlabeled Promegestone for determination of non-specific binding) for 4 h at 4° C.

All other parameters are as in the standard (nuclear) PR binding assay (described below): Following incubation in a final volume of 200 μl, the samples are filtered rapidly under vacuum though treated glass fibers and rinsed several times with ice-cold wash buffer, using a 96-sample cell harvester (Unifilter, Packard). The filters are dried then counted using a scintillation cocktail (Microscint O, Packard). Incubation buffer: 5 mM Na2HPO4/NaH2PO4 pH 7.4, 20 mM Na2MoO4, 10 mM monothioglycerol+10% glycerol. Stop buffer: 50 mM Tris/HCl pH7.4 Filtration on Unifilter GF/B pre-treated with 50 mM Tris/HCl pH7.4+0.3% PEI.

Positive Control: Standard test (see below) using a T47D cell preparation. One experiment (N=1) is performed in duplicate (n=2).

Based upon the results, one optimal quantity of NTERA-2 cell membranes is selected for the subsequent experiments.

### Phase 3: Assay Development and Compound Testing

Using the NTERA-2 cell membranes at the quantity selected in the previous phases, the following assay parameters are be optimized:
Test of 6 different incubation times: 0.5/1/2/3/4 h/overnight. One optimal incubation time is selected.
Test of 3 different incubation temperatures: 4/22/37° C. One optimal incubation temperature is selected.
Test of 12 different [³H]Progesterone concentrations: 0.1-10× the estimated Kd (if possible). Kd and Bmax are determined, and one optimal [³H]Progesterone concentration will be selected.
Test of 4 different DMSO concentrations: 0/0.1/0.3/1/3% final. The maximal DMSO concentration tolerated in the assay is determined.
All other conditions (buffers, filtration, etc) are as in the standard (nuclear) PR binding assay, as outlined above.

Using the assay parameters optimized throughout the different experiments (optimal membrane protein quantity, optimal radioligand concentration, optimal incubation time and temperature), test compounds are then be tested at each 8 concentrations in a competition binding assay against the [³H]Progesterone binding to the NTERA-2 cell membranes. The results are expressed as % inhibition of the control radioligand specific binding. Ki values are determined (if possible). Each one experiment (N=1) is performed in duplicate (n=2).

### Standard (Nuclear) Progesterone Receptor (PR) Radioligand Binding Assay

The standard (nuclear) progesterone receptor (PR) radioligand binding assay is described in the table below.

Human progesterone receptor (PF) (agonist radioligand)

| | |
|---|---|
| Purpose | Evaluation of the affinity of compounds for the |
| | progesterone receptor (PR) expressed in human T47D |
| | cells, determined in a radioligand binding assay. |
| Experimental | Fractions of cell cytosol (1.2 × 10⁵ cells) are incubated |
| protocol | for 20 h at 4° C. with 0.5 nM [³H]progesterone |
| | in the absence or presence of the test compound |
| | in a buffer containing 5 mM Na₂HPO₄/NaH₂PO₄ |
| | (pH 7.4), 20 MM NA₂MoO₄, 10 mM monothioglycerol |
| | and 10% glycerol. Nonspecific binding is determined in |
| | the presence of 1 μM promegestone. |
| | Following incubation, the samples are filtered rapidly |
| | under vacuum through glass fiber filters (GF/B, Packard) |
| | presoaked with 0.3% PEI and rinsed several times |
| | with ice-cold 50 mM Tris-HCl using a 96-sample |
| | cell harvester (Unifilter, Packard). The filters are dried |
| | then counted for radioactivity in a scintillation counter |
| | (Topcount, Packard) using a scintillation cocktail |
| | (Microscint O, Packard). |
| | The results are expressed as a percent inhibition |
| | of the control radioligand specific binding. |
| | The standard reference compound is promegestone, |
| | which is tested in each experiment at several |
| | concentrations to obtain a competition curve from which |
| | its IC₅₀ is calculated. |

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety.

The methods and processes illustratively described herein suitably may be practiced in differing orders of steps, and that they are not necessarily restricted to the orders of steps indicated herein or in the claims. Under no circumstances may the patent be interpreted to be limited to the specific examples or embodiments or methods specifically disclosed herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method of screening for a candidate GABA receptor trafficking modulator comprising the steps of:
a) contacting a test agent with a cell expressing at least one gamma-aminobutyric acid (GABA) receptor subunit;
b) measuring a membrane-associated amount of the at least one GABA receptor subunit of the cell;
c) comparing the membrane-associated amount of the at least one GABA receptor subunit in the cell contacted with the test agent with a membrane-associated amount of the at least one GABA receptor subunit in a cell not contacted with the test agent; and
wherein if the membrane-associated amount in the cell contacted with the test agent is greater than the membrane-associated amount in the cell not contacted with the test agent, the test agent is a candidate GABA receptor trafficking modulator.

2. The method of claim 1, wherein step b) comprises measuring:
(1) an amount of the at least one GABA receptor subunit that is located on the cell membrane;
(2) an amount of the at least one GABA receptor subunit that is incorporated into a GABA receptor;
(3) a ratio between a membrane-associated amount of the at least one GABA receptor subunit and a soluble amount of the at least one GABA receptor subunit;
(4) a rate of endocytosis of membrane-associated GABA receptors; or any combination of (1)-(4).

3. The method of claim 1, wherein step b) comprises performing a Western Blot assay or an immunohistochemistry assay.

4. A method of screening for a candidate GABA receptor trafficking modulator comprising the steps of:
a) contacting a test agent with a cell expressing at least one gamma-aminobutyric acid (GABA) receptor subunit;
b) measuring an expression level of the at least one GABA receptor subunit of the cell;
c) comparing the expression level of the at least one GABA receptor subunit of the cell contacted with the test agent with an expression level of the at least one GABA receptor subunit of a cell not contacted with the test agent; and
wherein if the expression level of the at least one GABA receptor subunit in the cell contacted with the test agent is greater than the expression level of the at least one GABA receptor subunit in the cell not contacted with the test agent, the test agent is a candidate GABA receptor trafficking modulator.

5. The method of claim 4, wherein step b) comprises measuring
(1) a total amount of the at least one GABA receptor subunit in the cell; and/or
(2) a total amount of a nucleic acid encoding the at least one GABA receptor subunit in the cell.

6. The method of claim 4, wherein step b) comprises performing a Western Blot assay or a Northern Blot assay.

7. A method of screening for a candidate GABA receptor trafficking modulator comprising the steps of:
a) contacting a test agent with a cell expressing at least one gamma-aminobutyric acid (GABA) receptor subunit;
b) measuring a phosphorylation level of the at least one GABA receptor subunit in the cell contacted with the test agent;
c) comparing the phosphorylation level of the at least one GABA receptor subunit in the cell contacted with the test agent with a phosphorylation level of the at least one GABA receptor subunit in a cell not contacted with the test agent; and
wherein if the phosphorylation level in the cell contacted with the test agent is greater than the phosphorylation level in the cell not contacted with the test agent, the test agent is a candidate GABA receptor trafficking modulator.

8. The method of claim 7, wherein the phosphorylation is protein kinase C (PKC)-mediated phosphorylation.

9. The method of claim 7, wherein the phosphorylation level of an α4 GABA subunit, a β3 GABA subunit, or a combination thereof is measured.

10. The method of claim 7, wherein the phosphorylation occurs at S408/409 of the β3 subunit and/or at S433 of the α4 subunit.

11. The method of claim 7, wherein step b) comprises measuring the phosphorylation level via a Western Blot assay employing an anti-phosphorylated subunit antibody.

12. The method of claim 1, wherein the at least one GABA receptor subunit is selected from a α1 subunit, a β2 subunit, a γ2 subunit, an α4 subunit, a β3 subunit, and a δ subunit, and any combination thereof.

13. The method of claim 1, wherein the at least one GABA receptor subunit comprises a combination of α1β2γ2 subunits or a combination of α4β3δ subunits.

14. The method of claim 1, wherein the GABA receptor is selected from a synaptic GABA receptor, an extrasynaptic GABA receptor, and a combination thereof.

15. The method of claim 14, wherein the synaptic GABA receptor comprises one or more subunits selected from an α1 subunit, a β2 subunit, and a γ2 subunit.

16. The method of claim 14, wherein the extrasynaptic GABA receptor comprises one or more subunits selected from an α4 subunit, a β3 subunit, and a δ subunit.

17. The method of claim 1, wherein the at least one GABA receptor subunit is encoded by (1) an endogenous gene, (2) an artificial expression construct, or (3) a combination thereof.

18. The method of claim 1, wherein the GABA receptor trafficking modulator is a natural or synthetic neuroactive steroid.

19. The method of claim 1, wherein the GABA receptor trafficking modulator is a membrane progesterone receptor (mPR) modulator.

20. The method of claim 1, wherein the GABA receptor trafficking modulator is a progesterone analog.

21. The method of claim 1, wherein the cell is a brain cell.

22. The method of claim 1, wherein the cell contacted with the test agent is a dentate gyms granule cell (DGGC).

23. A method of screening for a candidate GABA receptor trafficking modulator comprising the steps of:
a) contacting a test agent with a cell expressing at least one membrane progesterone receptor (mPR);
b) measuring an activity level of a mPR signaling pathway in the cell contacted with the test agent;
c) comparing the activity level of the mPR signaling pathway in the cell contacted with the test agent with an activity level of the mPR signaling pathway in a cell not contacted with the test agent;
wherein if the activity level of the mPR signaling pathway in the cell contacted with the test agent is greater than the activity level of the mPR signaling pathway in the cell not contacted with the test agent, the test agent is a candidate GABA receptor trafficking modulator.

24. The method of claim 23, wherein the greater activity level is indicated by an increase in protein kinase C (PKC) activity.

25. The method of claim 23, wherein the greater activity level is indicated by an increase in PKC-mediated phosphorylation of at least one GABA receptor subunit in the cell.

26. The method of claim 23, wherein the greater activity level is indicated by a reduced level of cellular cAMP.

27. The method of claim 23, wherein the greater activity level is indicated by an increase in a gene expression level, wherein the gene encodes for at least one GABA receptor subunit or the gene is a reporter gene.

28. The method of claim 27, wherein the gene is an endogenous gene or an artificial expression construct.

29. The method of claim 23, wherein the greater activity level is indicated by a higher membrane-associated amount of at least one GABA receptor subunit.

30. The method of claim 23, wherein the greater activity level is indicated by a greater GABAergic current in the cell.

31. The method of claim 23, wherein the greater activity level is indicated by an increase in association between the mPR and a substrate.

32. The method of claim 23, wherein step b) comprises measuring a level of
(1) PKC activity;
(2) PKC-mediated phosphorylation of at least one GABA receptor subunit;
(3) cellular cAMP;
(4) expression of a gene encoding for at least one GABA receptor subunit or a reporter gene;
(5) a membrane-associated amount of at least one GABA receptor subunit;
(6) GABAergic current in the cell; or
any combination of (1)-(6).

33. The method of claim 23, wherein the method further comprises d) measuring a binding affinity between the test agent and the mPR; and wherein if the binding affinity is above a predetermined threshold, the test agent is a candidate GABA receptor trafficking modulator.

34. The method of claim 1, further comprising a method of screening for a candidate GABA receptor potentiator, said method comprising:
d) contacting the test agent with a membrane-associated gamma-aminobutyric acid (GABA) receptor;
e) measuring a GABAergic current conducted by the GABA receptor of the membrane contacted with the test agent in the presence of GABA;
f) comparing the GABAergic current of conducted by the GABA receptor contacted with the test agent with a GABAergic current conducted by the GABA receptor not contacted with the test agent; and
wherein if the GABAergic current conducted by the GABA receptor contacted with the test agent is greater than the GABAergic current of conducted by the GABA receptor not contacted with the test agent, the test agent is a candidate GABA receptor potentiator.

35. The method of claim 34, wherein the GABA receptor is on a cell membrane.

36. The method of claim 34, wherein the GABA receptor is on a postsynaptic cell membrane.

37. The method of claim 34, wherein the GABA receptor is located within a synaptic area of the postsynaptic cell membrane.

38. The method of claim 34, wherein the GABA receptor is located outside a synaptic area of the postsynaptic cell membrane.

39. The method of claim 34, wherein the GABAergic current is a tonic current and/or a spontaneous inhibitory post-synaptic current (sIPSC).

40. The method of claim 39, wherein a greater GABAergic current is indicated by:
(1) a larger average amplitude of the tonic current;
(2) a higher average current density of the tonic current;
(3) a larger average amplitude of the sIPSC;
(4) a longer average decay time of the sIPSC; or
(5) any combination of (1)-(4).
